(19) United States
(12) Patent Application Publication
(10) Pub. No.: US 2003/0229044 A1
(43) Pub. Date: Dec. 11, 2003

(54) Use of statins and other immunomodulatory agents in the treatment of autoimmune disease

(76) Inventors: Lawrence Steinman, Stanford, CA (US); Hideki Garren, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/404,679

(22) Filed: Mar. 31, 2003

Related U.S. Application Data
(60) Provisional application No. 60/368,803, filed on Mar. 29, 2002.

(51) Int. Cl. A61K 48/00; A61K 38/16; A61K 31/401; A61K 31/366

(52) U.S. Cl. 514/44; 514/423; 514/460; 514/2

(57) ABSTRACT

Methods are provided for the treatment of autoimmune diseases, by co-administering a statin and a second immunomodulaotry agent. The second immunomodulatory agent can be antigen-specific or non-antigen-specific.

### CROSS-REFERENCES TO RELATED APPLICATIONS

This application derives priority from U.S. S No. 60/368,803, filed Mar. 29, 2002, which is incorporated by reference herein in its entirety for all purposes.

### STATEMENT AS TO RIGHTS TO INVENTIONS MADE UNDER FEDERALLY SPONSORED RESEARCH OR DEVELOPMENT

The research was supported at least in part by a grant from the National Institutes of Health, grant no. ROI NS 18235. The government may have certain rights in the invention.

### BACKGROUND OF THE INVENTION

The complexity of the immune system has been a daunting barrier to an understanding of immune system dysfunction. In recent years, the techniques of molecular biology have provided insight into the mechanisms and components that underlie immunity. To a large extent, the story of immunity is the story of lymphocytes. Lymphocytes possess an extremely complex and subtle system for interacting with each other, with antigen-presenting cells, and with foreign antigens and cells. Examples of autoimmune diseases include multiple sclerosis, rheumatoid arthritis, insulin dependent diabetes mellitus, autoimmune uveitis, and primary billiary cirrhosis.

Multiple Sclerosis (MS) is the most common central nervous system (CNS) demyelinating disease, affecting 350,000 (0.1%) individuals in North America and 1.1 million worldwide. In general, MS is considered to be an autoimmune disease mediated in part by proinflammatory CD4 T (Th1) cells that recognize specific myelin proteins in association with MHC class II molecules expressed on antigen (Ag) presenting cells (APC). Similar to other autoimmune diseases, MS susceptibility is genetically linked to the MHC HLA-D region (HLA DR2 (DRβ*1501, DQβ*0602).

MS is multiphasic. Attacks of neurologic impairment occur in the early phase, which is characterized histologically by inflammatory lesions containing a predominance of CD4 T cells, B cells and both MHC class II positive macrophages and microglia, a resident CNS antigen presenting cell (APC). After multiple acute attacks a chronic “secondary progressive” phase with sustained neurologic impairment often ensues. This “irreversible” phase is characterized by neuronal loss and atrophy.

In the U.S., two IFN-β medications, avonex (IFN-β 1a) and betaseron (IFN-β 1b), and copaxone (glatiramer acetate) have been approved for treatment of the early inflammatory “relapsing-remitting” phase. The IFN β's exert several effects in an Ag-nonspecific manner while copaxone appears to preferentially affect T cells specific for CNS autoantigens. Novantrone, a cancer chemotherapeutic agent that interferes with DNA repair, has been approved for treatment of secondary progressive MS. In addition to their side effects and potential toxicities, these medications are only partially effective, underscoring the need to develop new immunomodulatory MS therapies.

Approved for their cholesterol lowering effects in prevention of atherogenesis, evidence suggests that the 3-hydroxy-3-methylglutaryl coenzyme A (HMG-CoA) reductase inhibitors known as “statins” may be beneficial in treatment of inflammatory diseases. In 1995, it was reported that pravastatin treatment of cardiac transplant patients was associated with a reduction in hemodynamically significant rejection episodes and increased survival, independent of its cholesterol lowering effects. Metabolites of mevalonate, the product of HMG-CoA reductase, were known to be involved in post-translational modification (isoprenylation) of specific proteins involved in signal transduction and cell differentiation. However, greater appreciation for the potential immunodulatory effects of statins developed when it was demonstrated that lovastatin inhibited production of nitric oxide synthase (iNOS) and proinflammatory cytokines (TNFα, IL-1β and IL-6) by microglia and astrocytes, another CNS APC. The observation that statins inhibited iNOS secretion suggested they might also have neuroprotective effects.

Statins prevented IFN-γ-inducible class II expression on nonprofessional APC by inhibiting transcription at the IFN-γ-inducible promoter (p) pIV of the MHC class II transactivator (CIITA), the master regulator for class II expression, but did not alter constitutive expression in dendritic cells, which utilize pl or B cells, which use plll. Thus, statins may suppress Ag presentation by nonprofessional resident CNS APC.

Statins inhibit lymphocyte secretion of matrix metalloprotease-9 (MMP-9), an enzyme involved in basement membrane degradation and transmigration across endothelial barriers, including the blood brain barrier. Independent of HMG-CoA reductase inhibition, statins bind lymphocyte function-associated antigen-1 (LFA-1), a β32-integrin, and prevent interaction with its ligand, ICAM-1, and T cell activation. These observations suggest that statins may have beneficial effects at multiple steps in the pathogenic cascade of MS. In contrast with current MS treatments, which are administered parenterally, statins are given orally and are well tolerated. As statins appear to have different activities than currently approved MS treatments, they may also be useful in combination therapy, in addition to being considered as candidates for monotherapy.

Other examples of autoimmune diseases include rheumatoid arthritis, insulin dependent diabetes mellitus, autoimmune uveitis, and primary billiary cirrhosis.

Rheumatoid arthritis (RA) is a chronic autoimmune inflammatory synovitis affecting 0.8% of the world population. It is characterized by chronic inflammatory synovitis that causes erosive joint destruction. RA is mediated by T cells, B cells and macrophages.

Human type I or insulin-dependent diabetes mellitus (IDDM) is characterized by autoimmune destruction of the β cells in the pancreatic islets of Langerhans. The depletion of β cells results in an inability to regulate levels of glucose in the blood. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2), each an example of a self-protein, -polypeptide or -peptide according to this invention.

Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNFα-antagonists.

Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis.

### BRIEF SUMMARY OF THE INVENTION

The present invention provides methods for treating an autoimmune disease by co-administering to a patient suffering from the disease effective amounts of a statin and a second immunomodulatory agent. Autoimmune diseases that can be treated according to the methods provided herein include, for example, multiple sclerosis, insulin dependent diabetes mellitus (IDDM), rheumatoid arthritis, or autoimmune uveitis. The autoimmune disease can be multiphasic such as, for example, a demyelinating autoimmune disease (e.g., multiple sclerosis).

In certain embodiments, the statin is administered after the initial onset of the autoimmune disease. For example, the statin can be administered during a period of remission or during an active episode of the disease. The statin can be, for example, rosuvastatin, mevastatin, lovastatin, pravastatin, simvastatin, fluvastatin, atorvastatin, or cerivastatin.

In certain embodiments of the invention, the second immunomodulatory agent is antigen specific. In one embodiment, the antigen-specific immunomodulatory agent is a self-vector that includes a polynucleotide encoding a self-polypeptide associated with the autoimmune disease. The self-polypeptide encoded by the polynucleotide can be, for example, a protein or a peptide. In certain embodiments, the self-vector comprising a polynucleotide encodes one self-polypeptide.

In another embodiment, the antigen-specific immunomodulatory agent is a polypeptide. The polypeptide can be, for example, a protein or a peptide. In addition, the polypeptide can include a self-polypeptide associated with the disease or can include amino acids corresponding to an autoantigenic epitope of a self-polypeptide associated with the disease. In embodiments where the polypeptide includes amino acids corresponding to an autoantigenic epitope, the amino acids can be randomized to form a random copolymer or ordered such that the polypeptide includes an ordered amino acid motif. In one embodiment where the autoimmune disease is a demyelinating autoimmune disease, the ordered amino acid motif is [¹E²Y³Y⁴K]ₙ, where n is from 2 to 6.

In embodiments where a demyelinating autoimmune disease is treated (e.g., multiple sclerosis), the polypeptide encoded by the polynucleotide can be, for example, myelin basic protein (MBP), proteolipid protein (PLP), myelin associated glycoprotein (MAG), cyclic nucleotide phosphodiesterase (CNPase), myelin-associated oligodendrocytic basic protein (MBOP), myelin oligodendrocyte protein (MOG), or alpha-B crystalline. The demyelinating disease can be, e.g., multiple sclerosis.

In embodiments where insulin dependent diabetes mellitus is treated, the self-polypeptide encoded by the polynucleotide can be, for example, insulin, insulin B chain, preproinsulin, proinsulin, glutamic acid decarboxylase (65 kDa or 67 kDa forms), tyrosine phosphatase IA2 or IA-2b, carboxypeptidase H, a heat shock protein, glima38, the 69 kDa form of islet cell antigen, p52, or islet cell glucose transporter (GLUT 2). In certain embodiments, the self-vector comprising a polynucleotide encodes one self-polypeptide such as, for example, preproinsulin or insulin B chain 9-23.

In other embodiments, the autoimmune disease is rheumatoid arthritis. Where rheumatoid arthritis is treated, the polypeptide encoded by the polynucleotide can be, for example, type II collagen; hnRNP A2/RA33; Sa; filaggrin; keratin; cartilage proteins including gp39; collagens type I, III, IV, V, IX, XI; HSP-65/60; RNA polymerase; hnRNP-B1; hnRNP-D; or aldolase A.

In embodiments where autoimmune uveitis is treated, the polypeptide encoded by the polynucleotide can be, for example S-antigen, interphotoreceptor retinoid binding protein (IRBP), rhodopsin, or recoverin.

In other embodiments, the second immunomodulatory agent is non-antigen-specific. In one embodiment, the non-antigen specific immunomodulatory agent is ostepontin or a self-vector comprising a polynucleotide encoding osteopontin. In other embodiments, the non-antigen specific immunomodulatory agent is an immune modulatory sequence. The immune modulatory sequence can be, for example, 5′-Purine-Pyrimidine-[X]-[Y]-Pyrimidine-Pyrimidine-3′ or 5′-Purine-Purine-[X]-[Y]-Pyrimidine-Pyrimidine-3′, where X and Y are any naturally occurring or synthetic nucleotide, except that X and Y cannot be cytosine-guanine.

### BRIEF DESCRIPTION OF THE DRAWINGS

FIG. 1. EAE prevention and treatment by oral atorvastatin. Treatment at onset of MOG p35-55-induced EAE in C57B1/6 mice prevents clinical worsening (A, 7 mice in each group), while treatment after onset ameliorates EAE (B, 14 mice in each group). Treatment at onset of PLP p139-151-induced EAE in SJL/J mice prevents relapses (C, 10 mice in each group), while treatment begun after acute EAE reverses relapsing EAE (D, 10 mice in each group), prevention of acute EAE of MBP Ac1-11 induced in MBP Ac1-11 Tg mice (E, 6 mice in each group). Horizontal bars beneath each graph indicate atorvastatin treatment period. Mean EAE score are plotted against the number of days since EAE induction.

FIG. 2. Atorvastatin treatments decrease mononuclear infiltration in brains.

FIG. 3. Atorvastatin downregulates the expression of the different CIITA transcripts in vivo in the CNS. 3 groups of SJL mice were treated with: 1 mg/kg or 10 mg/kg atorvastatin or only PBS for 12 days. Two days after the beginning of the treatment EAE was induced in those mice using PLP139-151/CFA. At day 12 of the treatment (that equals day 10 of EAE) 2 mice of each group and two naive mice were sacrificed. Total RNA was extracted from the brains and total CIITA expression and specific CIITA expression was analyzed using Real Time PCR technique. (A) Shows the total expression of all CIITA mRNA transcripts (demonstrated as the internal transcripts), (B) shows the specific expression of the form I or as designed promoter I (PI, specific for dendritic cells), (C) shows the specific expression of the form III or as designed promoter III (P III, specific for B cells) and (D) shows the specific expression of the form IV or as designed promoter IV (P IV, the IFN-γ inducible form and specific for Microglia cells). Mean transcripts copies are plotted against the treated groups. Asterisks indicate a statistically significant difference (p 0.05 by one way ANOVA test) comparing the atorvastatin treated or naive groups versus the PBS treated group in each case.

FIG. 4. Atorvastatin suppresses proliferation and promotes Th2 cytokine bias. (A) Proliferative responses of PLP p139-151-stimulated spleen cells from atorvastatin-treated and vehicle-treated PLP p139-151 immunized mice. Atorvastatin treatment is associated with diminished secretion of IL-2 (b) and IFN-γ (c), and increased production of IL-4 (D) and IL-10 (at 10 mg/kg atorvastatin) (E) Proliferation was measured by ³H-thymidine incorporation, and cytokine measurements by ELISA.

FIG. 5. (A) An anti-phospho STAT6-specific Western was done in order to determine the extent of STAT6 activation in mice treated with PBS (lane 1), 1 mg/kg atorvastatin (lane 2), 10 mg (kg atorvastatin (lane 3), or mrIL-4 (10 ng/ml) treated lymphocytes (lane 4). Samples were obtained from protein lysates of draining lymph node cells from the different groups of mice. As seen in the positive control (lane 4) IL-4 treatments and Atorvastatin treatment s activate an expected 105 kDa isoform of STAT6 in lymph node cells. (B and C). The same blot was stripped and reprobed with antibodies against Stat6 (B) or mouse CD3 (C) as a control to ensure equal loading of each lane. The data shown are representative of two separate Western blots performed on each of two independent experiments. Molecular weights are indicated in kilodaltons.

FIG. 6A. DNA encoding a peptide from the self-protein proteolipid protein (PLP) reduces T cell proliferative responses. Lymph node cell (LNC) proliferative responses to PLP 139-151 were reduced in DNA vaccinated mice. After recovery from the acute phase of disease animals injected either with DNA coding for PLP139-151 (A) or control vector, pTarget (B) were sacrified and, draining LNC were isolated. Cells were tested in vitro by stimulation with different concentrations of the peptide PLP139-151 (squares) or the control peptide PLP178-191 (triangles). Proliferative responses from pooled LNC of groups of five animals are shown as mean CPM±SD of triplicate wells. CPM of Concanavalin A (0.001 mg/ml) stimulated LNC were 102401 for group A and 76702 for group B.

FIG. 6B. Cytokine levels are reduced in LNC from DNA immunized animals based on ELISA analysis. After the acute phase of EAE, LNC from groups of five animals vaccinated with either plasmid DNA coding for the PLP 139-151 or vector alone (pTarget), were stimulated in vitro with the immunizing peptide PLP 139-151. Levels of γ-interferon (striped bars) or IL-2 (dotted bars) were tested by ELISA in supernatants and compared to known standard controls. Results are expressed in ng/ml.

FIG. 6C. Cytokine levels are reduced in LNC from DNA immunized animals based on RNase Protection Assays. For cytokine mRNA detection, RNA samples from brains of experimental animals were tested using the Multi-Probe RNase Protection Assay and reactions were analyzed by 5% polyacrylamide gel electrophoresis. The gel was dried at the end of the run and exposed to x-ray film.

FIG. 7. Polynucleotide therapy with Inhibitory IMS suppresses PLP139-151 mediated EAE. On day 0, seven-week old female SJL/J mice were immunized subcutaneously with 100 μg PLP139-151 in PBS emulsified in CFA, consisting of IFA and 0.5 mg heat-inactivated Mycobacterium tuberculosis. Animals were clinically scored daily beginning on day 7. On day 12, mice were injected in both quadriceps with a total of 0.1 ml 0.25% Bupivacaine-HCL in PBS. Two days later, selected mice were injected intramuscularly in both quadriceps with DNA polynucleotide encoding full-length murine PLP, MAG, MOG, and MBP each on a separate pTARGET plasmid (25 μg of each) plus 50 μg pTARGET plasmid encoding full-length murine IL-4 in a total volume of 0.2 ml TE. DNA injections were given at weekly intervals for six weeks. At the same time as initial DNA treatment, 50 μg IMS in a volume of 200 μl PBS was administered intraperitoneally alone or with DNA polynucleotide treatment. IMS was given every other week for six weeks.

FIG. 8 is a graph depicting the prevention of EAE in rats treated with ordered peptides. Figure legend: Ordered peptide {SEQ ID NO:4} EYYKEYYKEYYK prevents the development of EAE in Lewis rats. Animals were injected with an emulsion of 0.1 mg of MBPp85-99 in complete Freund's adjuvant for EAE induction. Ten days later, when the clinical manifestations of disease became apparent, a single intra peritoneal dose of peptide {SEQ ID NO:4} EYYKEYYKEYYK (squares), {SEQ ID NO:5} KYYKYYKYYKYY (triangles), or PBS (circles) was administered. Results are expressed as mean disease score of groups of six animals.

FIG. 9A. Combination of 1 mg/kg atorvastatin and DNA encoding the self-protein proteolipid protein (PLP) reduces EAE severity. On day 0, seven-week old female SJL/J mice were immunized subcutaneously with 100 μg PLP139-151 in PBS emulsified in CFA, consisting of IFA and 0.5 mg heat-inactivated Mycobacterium tuberculosis. Animals were clinically scored daily beginning on day 7. On day 15, mice were injected in both quadriceps with a total of 0.1 ml 0.25% Bupivacaine-HCL in PBS. Two days later, mice were randomly divided into treatment groups and injected intramuscularly in both quadriceps with DNA polynucleotide encoding full-length murine PLP (50 μg per mouse) a total volume of 0.2 ml TE. DNA injections were given at weekly intervals throughout the experiment. At the same time as initial DNA treatment, atovastatin was administered orally in a volume of 0.5 ml at a dose of 1 mg/kg. Atorvastatin treatment was administered daily throughout the experiment. The control mice were given 0.5 ml of PBS orally on a daily basis. Mice were monitored daily for EAE disease and the mean scores for a treatment group are indicated.

FIG. 9B. Combination of 10 mg/kg atorvastatin and DNA encoding the self-protein proteolipid protein (PLP) reduces EAE severity. Experiments were conducted as in FIG. 1A with the exception of the atorvastatin administered at 10 mg/kg.

FIG. 9C. DNA treatment provides an equivalent benefit at both the 1 mg/kg and 10 mg/kg doses of atorvastatin. A direct comparison of the data in FIGS. 1A and 1B to demonstrate that the DNA effect is equivalent at the two doses of atorvastatin tested.

### DETAILED DESCRIPTION OF THE INVENTION

The methods of the present invention provide combined therapies for treating autoimmune disease, including multiphasic autoimmune disease such as autoimmune demyelinating disease (e.g., multiple sclerosis), using mevalonate pathway inhibitors such as, e.g., statins. The FDA has approved the long-term use of beta-interferons and glatiramer acetate, which is a synthetic form of myelin basic protein (MBP) that has fewer side effects than interferon. Other therapies include the administration of autoantigen encoding nucleic acids, peptides, and other immunosuppressive regimens. The combined use of other agents with mevalonate pathway inhibitors such as statins can have the advantages that the required dosages for the individual drugs is lower, and the effect of the different drugs complementary.

The combined therapy methods for treating autoimmune disease include co-administering to a patient suffering from the disease an effective dose of an inhibitor of mevalonate pathways and an effective dose of a second immunomodulatory agent. In preferred embodiments, the mevalonate pathway inhibitor is a statin. It is shown that statins switch the immune response to regulatory Th2 response, primarily through the production IL-4 and IL-10 cytokines, and are able to successfully reverse paralysis in relapsing demyelinating disease when treatment is initiated after the first attack.

In certain embodiments, the second immunomodulatory agent is antigen-specific. Preferred antigen-specific immunomodulatory agents include self-vectors, where the self-vector comprises a polynucleotide encoding a self-polypeptide associated with the disease. In other preferred embodiments, the autoimmune disease treated is a demyclinating autoimmune disease and the antigen-specific immunomodulatory agent is an ordered peptide that includes a repeated motif (SEQ ID NO: 1) [¹E²Y³Y⁴K]ₙ, where n is from 2 to 6.

In other embodiments of the invention, the second immunomodulatory agent is non-antigen-specific. In a preferred embodiment, the non-antigen-specific immunomodulatory agent is an immune modulatory oligonucleotide.

The active agents (mevalonate pathway inhibitor or immunomodulatory agent) may be administered before, during or after the onset of disease. While the subject methods are used for prophylactic or therapeutic purposes, of particular interest is the co-administration of mevalonate pathway inhibitor and antigen-specific immunomodulatory agent after onset of the disease, for example during remission; during a recurring disease incident; and the like. It is shown that a mevalonate pathway inhibitor in combination with an antigen-specific therapeutic agent can successfully reverse paralysis resulting from relapsing demyelinating disease, when treatment is initiated after the first attack.

Prior to setting forth the invention in more detail, it may be helpful to a further understanding thereof to set forth definitions of certain terms as used hereinafter.

Definitions:

Unless defined otherwise, all technical and scientific terms used herein have the same meaning as commonly understood by one of ordinary skill in the art to which this invention belongs. Although any methods and materials similar to those described herein can be used in the practice or testing of the present invention, only exemplary methods and materials are described. For the purposes of the present invention, the following terms are defined below.

The terms “a,” “an,” and “the” as used herein are not limiting and include plural referents unless the context clearly indicates otherwise. Thus, for example, reference to “a complex” includes a plurality of such complexes and reference to “the formulation” includes reference to one or more formulations and equivalents thereof known to those skilled in the art, and so forth.

As used herein, the term “treating” is used to refer to both prevention of disease and treatment of pre-existing conditions.

The term “autoimmune disease” refers to any disorder having a pathogenesis characterized at least in part by adaptive immunity that becomes misdirected at healthy cells and/or tissues of the body. Autoimmune diseases are characterized by T and/or B lymphocytes that aberrantly target self-molecules (e.g., self-polypeptides), causing injury and/or malfunction of an organ, tissue, or cell-type within the body (e.g., pancrease, brain, thyroid, or gastrointestinal tract). Autoimmune diseases include disorders that affect specific tissues as well as multiple tissues. Further, “autoimmune disease” as used herein can include acute, chronic, and/or relapsing-remitting forms of a disease. Examples of autoimmune diseases include rheumatoid arthritis, graft-versus host disease (GvHD), inflammatory bowel disease (IBD), insulin dependent diabetes mellitus (IDDM), multiple sclerosis, primary biliary cirrhosis, systemic sclerosis, psoriasis, autoimmune thyroiditis, and autoimmune thrombocytopenic purpura.

“Subject” or “patient” shall mean any animal, such as, for example, a human, non-human primate, horse, cow, dog, cat, mouse, rat, guinea pig, or rabbit.

The terms “molecule,” “compound,” and “agent” as used herein are synonymous and are used broadly to mean molecules that are potentially capable of structurally interacting with proteins through non-covalent interactions, such as, for example, through hydrogen bonds, ionic bonds, van der Waals attractions, or hydrophobic interactions. For example, agents most typically include functional groups necessary for structural interaction with proteins, particularly those groups involved in hydrogen bonding. Agents can include, for example, a small molecule drug; a peptide, including a variant analog, homolog, modified peptide or peptide-like substance such as a peptidomimetic or peptoid; or a protein a fragment thereof. An agent can be nonnaturally occurring, produced as a result of in vitro methods, or can be naturally occurring, such as, for example, a protein or fragment thereof expressed endogenously in a cell or from a cDNA library.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide typically having at least 10 contiguous amino acids, more typically at least 20, still more typically at least 50 contiguous amino acids of the polypeptide. A derivative is a polypeptide having conservative or non-conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural or “non-classical” amino acids, and the like), polypeptides with substituted linkages as well as other modifications known in the art, both naturally and non-naturally occurring. Thus, “polypeptide” can include a pharmaceutically acceptable salt of the polypeptide.

The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine.

“Self-polypeptide” as used herein refers to any polypeptide, or fragment or derivative thereof, that is encoded within the genome of the animal, is expressed in the animal, may be modified posttranslationally at some time during the life of the animal, and is associated with an autoimmune disorder as a self-antigen (i.e., autoantigen). Examples of posttranslational modifications of self-polypeptides are glycosylation, addition of lipid groups, dephosphorylation by phosphatases, addition of dimethylarginine residues, citrullination of fillagrin and fibrin by peptidyl arginine deiminase (PAD); alpha B crystallin phosphorylation; citrullination of MBP; and SLE autoantigen proteolysis by caspases and granzymes. “Antigen” refers to any molecule that can be specifically recognized by components of the immune response such as lymphocytes or antibodies. Self-polypeptide does not include immune proteins which are molecules expressed specifically and exclusively by cells of the immune system for the purpose of regulating immune function. Certain immune proteins that are included in the definition of self-polypeptide and they are: class I MHC membrane glycoproteins, class II MHC glycoproteins, and osteopontin.

“Self-vector” means a vector that includes a polynucleotide, either DNA or RNA, encoding a self-polypeptide. Polynucleotide, as used herein is a series of either deoxyribonucleic acids including DNA or ribonucleic acids including RNA, and their derivatives. The self-polypeptide-coding sequence is inserted into an appropriate plasmid expression self-cassette. Once the polynucleotide encoding the self-polypeptide, is inserted into the expression self-cassette the vector is then referred to as a “self-vector.” In the case where a polynucleotide encoding more than one self-polypeptide is to be administered, a single self-vector may encode multiple separate self-polypeptides. In one embodiment, DNA encoding several self-polypeptides are encoded sequentially in a single self-plasmid utilizing internal ribosomal re-entry sequences (IRES) or other methods to express multiple proteins from a single DNA molecule. The DNA expression self-vectors encoding the self-polypeptides are prepared and isolated using commonly available techniques for isolation of plasmid DNA such as those commercially available from Qiagen Corporation. The DNA is purified free of bacterial endotoxin for delivery to humans as a therapeutic agent. Alternatively, each self-polypeptide is encoded on a separate DNA expression vector. Self-vectors encompassed by the present invention are further defined in WO 053019.

“Plasmids” and “vectors” are designated by a lower case p followed by letters and/or numbers. The starting plasmids are commercially available, publicly available on an unrestricted basis, or can be constructed from available plasmids in accord with published procedures. In addition, equivalent plasmids to those described are known in the art and will be apparent to the ordinarily skilled artisan. A “vector” or “plasmid” refers to any genetic element that is capable of replication by comprising proper control and regulatory elements when present in a host cell. For purposes of this invention examples of vectors or plasmids include, but are not limited to, plasmids, phage, transposons, cosmids, virus, etc.

“Transfection” means introducing DNA into a host cell so that the DNA is expressed, whether functionally expressed or otherwise; the DNA may also replicate either as an extrachromosomal element or by chromosomal integration. Unless otherwise provided, the method used herein for transformation of the host cells is the calcium phosphate co-precipitation method of Graham and van der Eb (1973) Virology 52, 456-457. Alternative methods for transfection are electroporation, the DEAE-dextran method, lipofection and biolistics (Kriegler (1990) Gene Transfer and Expression: A Laboratory Manual, Stockton Press).

As used herein, “antigen-specific” in reference to an agent means that the agent can interact specifically with an antigen recognition molecule (e.g., T cell receptor, surface IgM on B cells) in such a way as to discriminate among antigen recognition molecules of the same class but having different antigenic specificities. Because antigen recognition molecules are typically clonally distributed among B or T lymphocytes, antigen-specific agents that are active can exert immunomodulatory effects on specific lymphocyte subsets expressing the particular antigen recognition molecule with which the agent interacts. Interaction of the agent with the antigen recognition molecule can be, for example, in the context of other molecular interactions, such as the binding of a peptide antigen to the T cell receptor as a peptide:MHC complex.

“Modulation of an immune response” as used herein refers to any alteration of an existing or potential immune response in vitro or in vivo. In the context of autoimmune disease, such alteration is of an immune response against self-molecules. Modulation can include any alteration in the presence or function of any immune cell (e.g., T cell, B cell, NK cell, macrophage, dendritic cell, neutrophil, mast cell, basophil, and the like) involved in or having the potential to be involved in the immune response. Modulation includes, for example, alteration in the expression and/or function of genes, proteins and/or other molecules in immune cells as part of an immune response; elimination, deletion, or sequestration of immune cells; induction or generation of immune cells that can modulate the functional capacity of other cells such as, e.g., autoreactive lymphocytes, antigen presenting cells (APCs), or inflammatory cells; induction of an unresponsive state in immune cells (e.g., anergy); or increasing, decreasing, or changing the activity or function of immune cells. Alteration in the pattern of proteins expressed by immune cells can include, for example, altered production and/or secretion of certain classes of molecules such as cytokines (e.g., IL-2, IFN-γ, TNF-α, IL-4), chemokines, growth factors, transcription factors (e.g., NF-κB), kinases (e.g. Lck, Lyn), phosphatases (e.g., PTP-1C, PTP-1D), costimulatory molecules (e.g., B7.1/B7.2, CTLA-4, CD40, ICAM, LFA-1), or other cell surface receptors.

“Immune Modulatory Sequences (IMSs)” as used herein refers to agents consisting of deoxynucleotides, ribonucleotides, or analogs thereof that modulate an autoimmune or inflammatory disease. IMSs may be oligonucleotides or a sequence of nucleotides incorporated in a vector. IMSs for use according to the methods provided herein are further described in U.S. patent application Ser. No. 10/302098, incorporated by reference herein in its entirety.

“Immunomodulatory agent” as used herein refers to a molecule that is capable of modulating a host's immune response. Immunomodulatory agents can be, for example, a nucleic acid (e.g., DNA) or a polypeptide (e.g., protein, glycoprotein, peptide, and the like). In addition, immunomodulatory agents can be antigen specific (e.g., a polypeptide that includes an autoantigenic epitope or that is immunologically cross-reactive with an autoantigenic epitope) or non-antigen-specific (e.g., cytokines, interleukins, interferons, or immune modulatory sequences). Immunomodulatory polypeptides can include recombinant or synthetic forms of a polypeptide. Immunomodulatory polypeptides can include, for example, polypeptides comprising autoantigens associated with the disease for which treatment is sought or, alternatively, a polypeptide that is immunologically cross reactive with the autoantigen. In addition, immunomodulatory polypeptides can include, for example, cytokines (or functional fragments thereof) such as, e.g., interleukins, interferons, or colony stimulating factors. Immunomodulatory polypeptides can also include, for example, chemokines or costimulatory molecules or functional fragments thereof. Where the native protein is a membrane bound molecule (e.g., receptors such as cytokine receptors (e.g., TNF-α R, IL-2R) or costimulatory molecules such as, for example, CD40, CTLA-4, or B7 molecules), the immunomodulatory polypeptide as used in the methods described herein can be a soluble form of the protein, such as, for example, an Ig fusion protein. Methods for making soluble Ig fusion recombinant forms of receptors are known in the art (see, e.g., U.S. Pat. No. 5,750,375).

The term “active agent” means any agent that can modulate an immune response.

The terms “effective amount” and “effective dose” as used herein are synonymous. An “effective dose” in context of administration of an agent (statin, self-vector, or ordered peptide) refers to an amount of a molecule that is sufficient to modulate an autoimmune response in a subject so as to inhibit the occurrence or ameliorate one or more symptoms of the target autoimmune response in a subject. Thus, an effective dose of an agent is the dose that, when administered for a suitable period of time, will evidence a reduction in the severity of the autoimmune disease. A suitable period of time for administration that will evidence a reduction in the severity of the autoimmune disease is usually at least about one week, and may be about two weeks, or more, up to a period of about 4 weeks. It will be understood by those of skill in the art that an initial dose may be administered for such periods of time, followed by maintenance doses, which, in some cases, will be at a reduced dosage.

Further, an effective amount of a statin, self-vector, or ordered peptide is administered according to the methods of the present invention in an “effective regime.” The term “effective regime” refers to a combination of amount of the agent and dosage frequency adequate to accomplish treatment or prevention of the autoimmune disease.

In addition, an effective amount of a particular agent in the context of combination therapy (e.g., statin plus self-vector or statin plus ordered peptide) means the amount of the particular agent that will, when co-administered for a suitable period of time with the second active agent, will evidence a reduction in the severity of the autoimmune disease as compared to that observed with the second active agent alone. In some embodiments, the combination of agents will produce a synergistic therapeutic effect. “Synergistic” as used herein means more than additive. In other embodiments of the invention, administration of an effective amount of the second active agent can reduce the amount of the first agent needed to evidence a reduction in the severity of the autoimmune disease as compared to that observed with the first agent alone. Such a reduction of the “effective amount” of an agent in the presence of a second active agent is herein referred to as “sparing,” i.e., the administration of the second agent spares the administration of the first agent.

Autoimmune Diseases

The present invention provides methods for treating or preventing autoimmune disease. Progression of disease can be measured by monitoring clinical or diagnostic symptoms using known methods such as, for example, methods described infra.

Several examples of autoimmune diseases that can be treated according to the methods provided herein are described below.

The methods of the invention are of particular interest for the treatment of demyelinating inflammatory diseases, which include multiple sclerosis, EAE, optic neuritis, acute transverse myelitis, and acute disseminated encephalitis.

Multiple Sclerosis. The course of disease for multiple sclerosis is highly varied, unpredictable, and, in most patients, remittent. The pathologic hallmark of MS is multicentric, multiphasic CNS inflammation and demyelination. Months or years of remission may separate episodes, particularly early in the disease. About 70% of patients of relapsing-remitting (RR) type, which is characterized by acute exacerbations with full or partial remissions. The remaining patients present with chronic progressive MS, which is subdivided further into (a) primary-progressive (PP), (b) relapsing-progressive (RP), which is a pattern combining features of RR and RP and is intermediate in clinical severity, and (c) secondary-progressive (SP), which many patients with RR progress to over time.

Clinical symptoms of MS include sensory loss (paresthesias), motor (muscle cramping secondary to spasticity) and autonomic (bladder, bowel, sexual dysfunction) spinal cord symptoms; cerebellar symptoms (e.g, Charcot triad of dysarthna, ataxia, tremor); fatigue and dizziness; impairment in information processing on neuropsychological testing; eye symptoms, including diplopia on lateral gaze; trigeminal neuralgia; and optic neuritis.

The autoantigen in MS most likely is one of several myelin proteins (e.g, proteolipid protein (PLP); myelin oligodendrocyte glycoprotein (MOG); myeline basic protein (MBP); myelin-associated glycoprotein (MAG), myelin-associated oligodendrocytic basic protein (MBOP); citrulline-modified MBP (the C8 isoform of MBP in which 6 arginines have been de-imminated to citrulline), cyclic nucleotide phosphodiesterase (CNPase), alpha-B crystalline, etc.) The integral membrane protein PLP is a dominant autoantigen of myelin. Microglial cells and macrophages perform jointly as antigen-presenting cells, resulting in activation of cytokines, complement, and other modulators of the inflammatory process, targeting specific oligodendroglia cells and their membrane myelin. A quantitative increase in myelin-autoreactive T cells with the capacity to secrete IFN-γ is associated with the pathogenesis of MS and EAE, suggesting that autoimmune inducer/helper T lymphocytes in the peripheral blood of MS patients may initiate and/or regulate the demyelination process in patients with MS.

Rheumatoid Arthritis. Rheumatoid arthritis (RA) is a chronic autoimmune inflammatory synovitis affecting 0.8% of the world population. It is characterized by chronic inflammatory synovitis that causes erosive joint destruction. RA is mediated by T cells, B cells and macrophages.

Evidence that T cells play a critical role in RA includes the (1) predominance of CD4+ T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the β chain that are involved in peptide binding and presentation to T cells. RA is mediated by autoreactive T cells that recognize a self-protein, or modified self-protein, present in synovial joints. Self-polypeptides of this invention also referred to as autoantigens are targeted in RA and comprise epitopes from type II collagen; hnRNP; A2/RA33; Sa; filaggrin; keratin; citrulline; cartilage proteins including gp39; collagens type I, III, IV, V, IX, XI; HSP-65/60; IgM (rheumatoid factor); RNA polymerase; hnRNP-B 1; hnRNP-D; cardiolipin; aldolase A; citrulline-modified filaggrin and fibrin. Autoantibodies that recognize filaggrin peptides containing a modified arginine residue (de-iminated to form citrulline) have been identified in the serum of a high proportion of RA patients. Autoreactive T and B cell responses are both directed against the same immunodominant type II collagen (CII) peptide 257-270 in some patients.

Insulin Dependent Diabetes Mellitus. Human type I or insulin-dependent diabetes mellitus (IDDM) is characterized by autoimmune destruction of the [cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2), each an example of a self-protein, -polypeptide or -peptide according to this invention.

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the [cells, which leads to hyperglycemia and overt diabetes. Both CD4+ and CD8+ T cells are required for diabetes to develop, although the roles of each remain unclear. It has been shown that administration of insulin or GAD, as proteins, under tolerizing conditions to NOD mice prevents disease and down-regulates responses to the other self-antigens.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens including GAD, insulin and IA-2 conveys a positive predictive value of >90% for development of type I DM within 5 years.

Autoantigens targeted in human insulin dependent diabetes mellitus may include the self-polypeptides tyrosine phosphatase IA-2; IA-2β; glutamic acid decarboxylase (GAD) both the 65 kDa and 67 kDa forms; carboxypeptidase H; insulin; proinsulin; heat shock proteins (HSP); glima 38; islet cell antigen 69 KDa (ICA69); p52; two ganglioside antigens (GT3 and GM2-1); and an islet cell glucose transporter (GLUT 2).

Human IDDM is currently treated by monitoring blood glucose levels to guide injection, or pump-based delivery, of recombinant insulin. Diet and exercise regimens contribute to achieving adequate blood glucose control.

Autoimmune Uveitis. Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNFα-antagonists.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Self-proteins targeted by the autoimmune response in human autoimmune uveitis may include S-antigen, interphotoreceptor retinoid binding protein (IRBP), rhodopsin, and recoverin.

Primary Billiary Cirrhosis. Primary Biliary Cirrhosis (PBC) is an organ-specific autoimmune disease that predominantly affects women between 40-60 years of age. The prevalence reported among this group approaches I per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol Rev 174:210-225, 2000); (Mackay et al., Immunol Rev 174:226-237, 2000). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-polypeptide of the instant invention. Studies identifying the role of pyruvate dehydrogenase complex (PDC) antigens in the etiopathogenesis of PBC support the concept that PDC plays a central role in the induction of the disease (Gershwin et al., Immunol Rev 174:210-225, 2000); (Mackay et al., Immunol Rev 174:226-237, 2000). The most frequent reactivity in 95% of cases of PBC is the E274 kDa subunit, belonging to the PDC-E2. There exist related but distinct complexes including: 2-oxoglutarate dehydrogenase complex (OGDC) and branched-chain (BC) 2-OADC. Three constituent enzymes (E1,2,3) contribute to the catalytic function which is to transform the 2-oxoacid substrate to acyl co-enzyme A (CoA), with reduction of NAD+ to NADH. Mammalian PDC contains an additional component, termed protein X or E-3 Binding protein (E3BP). In PBC patients, the major antigenic response is directed against PDC-E2 and E3BP. The E2 polypeptide contains two tandemly repeated lipoyl domains, while E3BP has a single lipoyl domain. The lipoyl domain is found in a number of autoantigen targets of PBC and is referred to herein as the “PBC lipoyl domain.” PBC is treated with glucocorticoids and immunosuppressive agents including methotrexate and cyclosporin A.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J Clin Pathol 53:813-21, 2000).

Other Autoimmune Diseases And Associated Self-Polypeptides. Autoantigens for myasthenia gravis may include epitopes within the acetylcholine receptor. Autoantigens targeted in pemphigus vulgaris may include desmoglein-3. Sjogren's syndrome antigens may include SSA (Ro); SSB (La); and fodrin. The dominant autoantigen for pemphigus vulgaris may include desmoglein-3. Panels for myositis may include tRNA synthetases (e.g., threonyl, histidyl, alanyl, isoleucyl, and glycyl); Ku; Scl; SSA; U1 Sn ribonuclear protein; Mi-1; Mi-1; Jo-1; Ku; and SRP. Panels for scleroderma may include Scl-70; centromere; U1 ribonuclear proteins; and fibrillarin. Panels for pernicious anemia may include intrinsic factor; and glycoprotein beta subunit of gastric H/K ATPase. Epitope Antigens for systemic lupus erythematosus (SLE) may include DNA; phospholipids; nuclear antigens; Ro; La; U1 ribonucleoprotein; Ro60 (SS-A); Ro52 (SS-A); La (SS-B); calreticulin; Grp78; Scl-70; histone; Sm protein; and chromatin, etc. For Grave's disease epitopes may include the Na+/I-symporter; thyrotropin receptor; Tg; and TPO.

Mevalonate Pathway Inhibitors

The methods according to the present invention for treating autoimmune disease comprise the use of agents that are inhibitors of mevalonate synthesis or effector pathways. Mevalonate metabolites are involved in modification of G-proteins, such as Ras. Inhibitors of the mevalonate pathway have been used to inhibit isoprenylation of Ras proteins and the Raf/MAP kinase cascade (Kikuchi et al., J. Biol. Chem., 269:20054-20059 (1994)). HMG-CoA reductase and mevalonate pyrophosphate decarboxylase are two useful targets for inhibition in the mevalonate pathway.

Statins. In a preferred embodiment, the mevalonate pathway inhibitor is a statin. Statins refer to a known class of of HMG-CoA reductase inhibitors. These agents are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520. Recently the “super statin” rosuvastatin has been commercialized. The lipophilicity of certain statins make them particularly suitable for subcutaneous delivery.

The data provided herein demonstrate that statins promote development of Th2 regulatory T cells by differentially altering the activation of specific STAT (signal transducers and activators of transcription) proteins. STATs are a family of transcription factors that are activated by phosphorylation via the Janus Kinase (JAK) family of tyrosine kinases. STAT6 is critical for Th2 differentiation while STAT4 has a pivotal role in Th1 differentiation. Statins differentially promote activation of STAT6 by phosphorylation. Statins also inhibit the regulation of the different CIITA promoters in the treated brain, and affect the inhibited CIITA-directed class II upregulation on nonprofessional CNS APC, thereby and preventing Ag presentation to Th1 cells.

The formulation and administration of statins is well known, and will generally follow conventional usage. The dosage required to treat autoimmune disease may vary from the levels used for management of cholesterol, and in some instances will be higher doses, around about 5 fold increase over conventional dosage (where conventional dosage is intended to refer to approved dosage for management of cholesterol); around about 10 fold increase over conventional dosage, and may be as much as 20 fold increase, or more.

The statins can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

Antigen-Specific Immunomodulatory Agents

In certain embodiments of the invention, antigen-specific immunomodulatory agents are co-administered with the mevalonate pathway inhibitor (e.g., statin). Antigen-specific immunomodulatory agents can be, for example, nucleic acids (e.g., DNA or RNA), such as a polynucleotide encoding an autoantigenic self-polypeptide associated with the disease. In addition, the antigen-specific immunomodulatory agent can be, e.g., a polypeptide. For example, the antigen-specific agent can be a polypeptide that includes an autoantigenic epitope associated with the disease. Such polypeptide can be, e.g., a protein autoantigen or a polypeptide that includes a fragment thereof (e.g., peptide fragment), the polypeptide fragment having the autoantigenic epitope. Also, for example, the polypeptide agent can include amino acids that are not identical to those constituting an autoantigenic epitope but immunologically cross-reactive with the epitope. In addition, the polypeptide can, for example, include amino acids corresponding to those of an autoantigenic epitope and, e.g., arranged randomly (a random copolymer such as, e.g., glatiramer acetate) or arranged as an ordered motif. Antigen-specific immunomodulatory polypeptides are at least about 6 amino acids in length, typically from about 6 to about 100 amino acids, more typically from about 8 to about 50 amino acids, and most typically from about 8 to about 25 amino acids. In other embodiments, the antigen specific immunomodulatory polypeptide can be a derivative polypeptide. A derivative is a polypeptide having conservative or non-conservative amino acid substitutions, as compared with another sequence. Derivatives further include, for example, glycosylations, acetylations, phosphorylations, and the like. Example of such derivatives include altered peptide ligands as described for example, U 6669033, 6322949.

Polynucleotides Encoding Self-Polypeptides. In certain embodiments of the invention, the antigen-specific immunomodulatory agent is a self-vector comprising a polynucleotide, where the polynucleotide encodes an autoantigenic self-polypeptide associated with the disease. The self-vectors are expression “self-cassettes” designed to express the encoded self-polypeptide when transfected into host cells.

Construction of the vectors of the invention employs standard ligation and restriction techniques which are well understood in the art (see Ausubel et al., (1987) Current Protocols in Molecular Biology, Wiley-Interscience or Maniatis et al., (1992) in Molecular Cloning: A laboratory Manual, Cold Spring Harbor Laboratory, N.Y.). Isolated plasmids, DNA sequences, or synthesized oligonucleotides are cleaved, tailored, and relegated in the form desired. The sequences of all DNA constructs incorporating synthetic DNA were confirmed by DNA sequence analysis (Sanger et al. (1977) Proc. Natl. Acad. Sci. 74, 5463-5467).

The expression self-cassette will employ a promoter that is functional in host cells. In general, vectors containing promoters and control sequences that are derived from species compatible with the host cell are used with the particular host cell. Promoters suitable for use with prokaryotic hosts illustratively include the beta-lactamase and lactose promoter systems, alkaline phosphatase, the tryptophan (trp) promoter system and hybrid promoters such as tac promoter. However, other functional bacterial promoters are suitable. In addition to prokaryotes, eukaryotic microbes such as yeast cultures may also be used. Saccharomyces cerevisiae, or common baker's yeast is the most commonly used eukaryotic microorganism, although a number of other strains are commonly available. Promoters controlling transcription from vectors in mammalian host cells may be obtained from various sources, for example, the genomes of viruses such as: polyoma, simian virus 40 (SV40), adenovirus, retroviruses, hepatitis B virus and preferably cytomegalovirus, or from heterologous mammalian promoters, e.g., β-actin promoter. The early and late promoters of the SV 40 virus are conveniently obtained as an SV40 restriction fragment which also contains the SV40 viral origin of replication. The immediate early promoter of the human cytomegalovirus is conveniently obtained as a HindIII restriction fragment. Of course, promoters from the host cell or related species also are useful herein.

The vectors used herein may contain a selection gene, also termed a selectable marker. A selection gene encodes a protein, necessary for the survival or growth of a host cell transformed with the vector. Examples of suitable selectable markers for mammalian cells include the dihydrofolate reductase gene (DHFR), the ornithine decarboxylase gene, the multi-drug resistance gene (mdr), the adenosine deaminase gene, and the glutamine synthase gene. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. The second category is referred to as dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern and Berg (1982) J. Molec. Appl. Genet. 1, 327), mycophenolic acid (Mulligan and Berg (1980) Science 209, 1422), or hygromycin (Sugden et al. (1985) Mol. Cell. Bio. 5, 410-413). The three examples given above employ bacterial genes under eukaryotic control to convey resistance to the appropriate drug neomycin (G418 or genticin), xgpt (mycophenolic acid) or hygromycin, respectively.

Self-vectors of this invention can be formulated as polynucleotide salts for use as pharmaceuticals. Polynucleotide salts can be prepared with non-toxic inorganic or organic bases. Inorganic base salts include sodium, potassium, zinc, calcium, aluminum, magnesium, etc. Organic non-toxic bases include salts of primary, secondary and tertiary amines, etc. Such self-DNA polynucleotide salts can be formulated in lyophilized form for reconstitution prior to delivery, such as sterile water or a salt solution. Alternatively, self-DNA polynucleotide salts can be formulated in solutions, suspensions, or emulsions involving water- or oil-based vehicles for delivery. In one preferred embodiment, the DNA is lyophilized in phosphate buffered saline with physiologic levels of calcium (0.9 mM) and then reconstituted with sterile water prior to administration. Alternatively the DNA is formulated in solutions containing higher quantities of Ca++, between 1 mM and 2M. The DNA can also be formulated in the absence of specific ion species.

As known to those ordinarily skilled in the art, a wide variety of methods exist to deliver polynucleotide to subjects, as defined herein. The polynucleotide encoding a self-polypeptide can be formulated with cationic polymers including cationic liposomes. Other liposomes also represent effective means to formulate and deliver self-polynucleotide. Alternatively, the self DNA can be incorporated into a viral vector, viral particle, or bacterium for pharmacologic delivery. Viral vectors can be infection competent, attenuated (with mutations that reduce capacity to induce disease), or replication-deficient. Methods utilizing self-DNA to prevent the deposition, accumulation, or activity of pathogenic self proteins may be enhanced by use of viral vectors or other delivery systems that increase humoral responses against the encoded self-protein. In other embodiments, the DNA can be conjugated to solid supports including gold particles, polysaccharide-based supports, or other particles or beads that can be injected, inhaled, or delivered by particle bombardment (ballistic delivery).

Methods for delivering mucleic acid preparations are known in the art. See, e.g., U.S. Pat. Nos. 5,399,346, 5,580,859, 5,589,466. A number of viral based systems have been developed for transfer into mammalian cells. For example, retroviral systems have been described (U.S. Pat. No. 5,219,740; Miller et al., Biotechniques 7:980-990, 1989; Miller, A. D., Human Gene Therapy 1:5-14, 1990; Scarpa et al., Virology 180:849-852, 1991; Burns et al., Proc. Natl. Acad. Sci. USA 90:8033-8037, 1993; and, Boris-Lawrie and Temin, Cur. Opin. Genet. Develop. 3:102-109, 1993). A number of adenovirus vectors have also been described, see e.g., (Haj-Ahmad et al., J. Virol. 57:267-274, 1986; Bett et al., J. Virol. 67:5911-5921, 1993; Mittereder et al., Human Gene Therapy 5:717-729, 1994; Seth et al., J. Virol. 68:933-940, 1994; Barr et al., Gene Therapy 1:51-58, 1994; Berkner, K. L., BioTechniques 6:616-629, 1988; and, Rich et al., Human Gene Therapy 4:461-476, 1993). Adeno-associated virus (AAV) vector systems have also been developed for nucleic acid delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., Molec. Cell. Biol. 8:3988-3996, 1988; Vincent et al., Vaccines 90 (Cold Spring Harbor Laboratory Press) 1990; Carter, B. J., Current Opinion in Biotechnology 3:533-539, 1992; Muzyczka, N., Current Topics in Microbiol. And Immunol. 158:97-129, 1992; Kotin, R. M., Human Gene Therapy 5:793-801, 1994; Shelling et al., Gene Therapy 1:165-169, 1994; and, Zhou et al., J. Exp. Med. 179:1867-1875, 1994).

The polynucleotide of this invention can also be delivered without a viral vector. For example, the molecule can be packaged in liposomes prior to delivery to the subject. Lipid encapsulation is generally accomplished using liposomes which are able to stably bind or entrap and retain nucleic acid. For a review of the use of liposomes as carriers for delivery of nucleic acids, see, (Hug et al., Biochim. Biophys. Acta. 1097:1-17, 1991; Straubinger et al., in Methods of Enzymology, Vol. 101, pp. 512-527, 1983).

“Therapeutically effective amounts” of the self-vector comprising a polynucleotide encoding a self-polypeptide is administered in accord with the teaching of this invention and will be sufficient to treat or prevent the disease as for example by ameliorating or eliminating symptoms and/or the cause of the disease. For example, therapeutically effective amounts fall within broad range(s) and are determined through clinical trials and for a particular patient is determined based upon factors known to the ordinarily skilled clinician including the severity of the disease, weight of the patient, age and other factors. Therapeutically effective amounts of self-vector are in the range of about 0.001 micrograms to about 1 gram. A preferred therapeutic amount of self-vector is in the range of about 10 micrograms to about 5 milligrams. A most preferred therapeutic amount of self-vector is in the range of about 0.025 mg to 5 mg. Polynucleotide therapy is delivered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the self-polypeptide being administered and such other factors as would be considered by the ordinary treating physician.

In one embodiment the polynucleotide is delivered by intramuscular injection. In another embodiment the polynucleotide is delivered intranasally, orally, subcutaneously, intradermally, intravenously, mucosally, impressed through the skin, or attached to gold particles delivered to or through the dermis (see e.g. WO 97/46253). Alternatively, nucleic acid can be delivered into skin cells by topical application with or without liposomes or charged lipids (see e.g. U.S. Pat. No. 6,087,341). Yet another alternative is to deliver the nucleic acid as an inhaled agent. The polynucleotide is formulated in phosphate buffered saline with physiologic levels of calcium (0.9 mM). Alternatively the polynucleotide is formulated in solutions containing higher quantities of Ca++, between 1 mM and 2M. The polynucleotide may be formulated with other cations such as zinc, aluminum, and others. Alternatively, or in addition, the polynucleotide may be formulated either with a cationic polymer, cationic liposome-forming compounds, or in non-cationic liposomes. Examples of cationic liposomes for DNA delivery include liposomes generated using 1,2-bis(oleoyloxy)-3-(trimethylammionio) propane (DOTAP) and other such molecules.

Prior to delivery of the polynucleotide, the delivery site can be preconditioned by treatment with bupivicane, cardiotoxin or another agent that may enhance the delivery of subsequent polynucleotide therapy. Such preconditioning regimens are generally delivered 12 to 96 hours prior to delivery of therapeutic polynucleotide, more frequently 24 to 48 hours prior to delivery of the therapeutic DNA. Alternatively, no preconditioning treatment is given prior to DNA therapy.

In addition to the self-vector encoding self-polypeptide, an adjuvant for modulating the immune response consisting of CpG oligonucleotides may be co-administered in order to enhance the immune response. CpG oligonucleotides have been shown to enhance the antibody response of DNA vaccinations (Krieg et al., Nature 374:546-9, 1995). The CpG oligonucleotides will consist of a purified oligonucleotide of a backbone that is resistant to degradation in vivo such as a phosphorothioated backbone. The specific sequence contained within the oligonucleotide will be purine-purine-C-G-pyrimidine-pyrimidine or purine-pyrimidine-C-G-pyrimidine-pyrimidine. All of these constructs will be administered in a manner such that an immune response is generated against the encoded self-polypeptide. The immune response, typically an antibody response, will affect the non-physiological action or process associated with the self-polypepetide.

Nucleotide sequences selected for use in the present invention can be derived from known sources, for example, by isolating the nucleic acid from cells containing a desired gene or nucleotide sequence using standard techniques. Similarly, the nucleotide sequences can be generated synthetically using standard modes of polynucleotide synthesis that are well known in the art. See, e.g., (Edge et al., Nature 292:756 1981); (Nambair et al., Science 223:1299 1984); (Jay et al., J. Biol. Chem. 259:6311 1984). Generally, synthetic oligonucleotides can be prepared by either the phosphotriester method as described by (Edge et al., (supra) and (Duckworth et al., Nucleic Acids Res. 9:1691 1981), or the phosphoramidite method as described by (Beaucage et al., Tet. Letts. 22:1859 1981), and (Matteucci et al., J. Am. Chem. Soc. 103:3185 1981). Synthetic oligonucleotides can also be prepared using commercially available automated oligonucleotide synthesizers. The nucleotide sequences can thus be designed with appropriate codons for a particular amino acid sequence. In general, one will select preferred codons for expression in the intended host. The complete sequence is assembled from overlapping oligonucleotides prepared by standard methods and assembled into a complete coding sequence. See, e.g., Edge et al. (supra); Nambair et al. (supra) and Jay et al. (supra).

Another method for obtaining nucleic acid sequences for use herein is by recombinant means. Thus, a desired nucleotide sequence can be excised from a plasmid carrying the nucleic acid using standard restriction enzymes and procedures. Site specific DNA cleavage is performed by treating with the suitable restriction enzymes and procedures. Site specific DNA cleavage is performed by treating with the suitable restriction enzyme (or enzymes) under conditions which are generally understood in the art, and the particulars of which are specified by manufacturers of commercially available restriction enzymes. If desired, size separation of the cleaved fragments may be performed by polyacrylamide gel or agarose gel electrophoreses using standard techniques.

Yet another convenient method for isolating specific nucleic acid molecules is by the polymerase chain reaction (PCR). (Mullis et al., Methods Enzymol. 155:335-350 1987).

Ordered Peptides. In certain embodiments of the invention, the antigen-specific immunomodulatory agent is a peptide having an ordered amino acid motif, where the amino acids correspond to amino acids in an antigenic epitope.

In one preferred embodiment for the treatment of demyelinating autoimmune disease, the ordered peptide comprise the ordered amino acid motif {SEQ ID NO: 1} [¹E²Y³Y⁴K]ₙ, where n is from 2 to 6. The ordered motif may start at residue 1, as shown, or may start at a different position, e.g. {SEQ ID NO:6} YYKEYYKEYYKE; {SEQ ID NO:7} KEYYKEYYKEYY, etc. The total length of the ordered peptide sequence will usually be at least about 8 amino acids in length and not more than about 24 amino acids in length, usually at least about 10 and not more than about 20. Specific peptides of interest include the sequence {SEQ ID NO:4} EYYKEYYKEYYK. The peptide may consist only of the ordered repeats, or may be extended at either termini by the addition of other amino acid residues.

Modification and changes may be made in the structure of the ordered peptide and still obtain a molecule having the desired characteristic of suppressing demyelinating autoimmune disease. The desired properties may be determined, at least in part, in an in vitro assay, where binding to the MHC antigen HLA-DR, particularly HLA-DR2 (DRB1*1501), is indicative of the relevant biological activity.

For example, certain amino acids may be substituted for other amino acids in a protein structure without appreciable loss of function. It will be understood by one of skill in the art that various changes (such as to protein stability or efficiency) may be made in the sequence of the ordered peptide without appreciable loss of their biological utility or activity, particularly as to the additional of terminal amino acids. So long as a change maintains the binding properties and immunological activity, the resultant protein will be considered a biologically functional equivalent for the purposes of the invention.

The peptides may be provided in a variety of ways, being joined to non-wild-type flanking regions, as fused proteins, joined by linking groups or directly covalently linked through cysteine (disulfide) or peptide linkages. The peptides may be joined to a single amino acid at the N- or C-terminus or a chain of amino acids. The fused peptides may be extended to provide convenient linking sites, e.g. cysteine or lysine, to enhance stability, to bind to particular receptors, to provide for site-directed action, to provide for ease of purification, to alter the physical characteristics (e.g. solubility, charge, etc.), to stabilize the conformation, etc. The peptide may be N-terminal, C-terminal or internal in relation to these added sequences.

The peptide may be linked through a variety of bifunctional agents, such as male imidobenzoic acid, methyldfhioacetic acid, mercaptobenzoic acid, S-pyridyl dithiopropionate, etc. The oligopeptides may be linked to proteins to provide site-directed action. The oligopeptides may be linked, particularly by an intracellular cleavable linkage, to antibodies for site directed action. For conjugation techniques, see, for example, U.S. Pat. Nos. 3,817,837; 3,853,914; 3,850,752; 3,905,654; 4,156,081; 4,069,105; and 4,043,989, which are incorporated herein by reference. The oligopeptides may also be modified by incorporation into the lumen of vesicles, e.g. liposomes, which in turn may be bound to ligands or receptors for direction to particular cells or tissue.

For therapy, the peptides may be administered topically or parenterally, e.g. by injection at a particular site, including subcutaneously, intraperitoneally, intravasculady, or the like or transdermally, as by electrotransport. In a preferred embodiment, subcutaneous injection is used to deliver the peptide. The oligopeptides may also be administered in a sustained release formulation or osmotic pump, to provide a depot of active peptide for slow release over an extended period. Such delivery may decrease the dosage of drug required and may also decrease the number of treatments necessary to achieve a therapeutic effect.

The oligopeptides of this invention may be prepared in accordance with conventional techniques, such as synthesis, recombinant techniques, or the like. For example, solid phase peptide synthesis involves the successive addition of amino acids to create a linear peptide chain (see Merrifield (1963) J. Am. Chem. Soc. 85:2149-2154). Production of the peptide by recombinant DNA technology may also be performed. One first synthesizes or otherwise creates a nucleic acid sequence that encodes the desired peptide. This coding sequence is operably connected to suitable control elements for expression, e.g. promoters, terminators, ATG start codon, and the like as known in the art. This expression construct is introduced into a suitable host cell, and the recombinant protein that is produced is isolated. Alternatively, the coding sequence is introduced into the host to be treated for long term therapy, for example by inserting an expression construct into muscle or long lived hematopoietic cells for therapy. The expression vector may be a plasmid, viral vector, including retrovirus, adenovirus, etc., and may be introduced by transduction, DNA vaccination, etc.

Pharmaceutically acceptable salts of the peptides also fall within the scope of the peptides as disclosed herein.

Various methods for administration may be employed. The formulation may be given orally, by inhalation, or may be injected, e.g. intravascular, intratumor, subcutaneous, intraperitoneal, intramuscular, etc. The dosage of the therapeutic formulation will vary widely, depending upon the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc. to maintain an effective dosage level. In many cases, oral administration will require a higher dose than if administered intravenously.

The peptides of the invention can be incorporated into a variety of formulations for therapeutic administration. More particularly, the complexes can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the peptides can be achieved in various ways, including oral, buccal, rectal, parenteral, intraperitoneal, intradermal, transdermal, intracheal, etc., administration. The peptides may be systemic after administration or may be localized by the use of an implant that acts to retain the active dose at the site of implantation.

In pharmaceutical dosage forms, the peptides may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the peptides can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The peptides can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The peptides can be utilized in aerosol formulation to be administered via inhalation. The compounds of the present invention can be formulated into pressurized acceptable propellants such as dichlorodifluoromethane, propane, nitrogen and the like.

Furthermore, the peptides can be made into suppositories by mixing with a variety of bases such as emulsifying bases or water soluble-bases. The peptides of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres; slabs, etc. with biodegradable or non-biodegradable polymers. For example, polymers of lactic acid and/or glycolic acid form an erodible polymer that is well-tolerated by the host. The implant containing peptides is placed in proximity to the site of action, so that the local concentration of active agent is increased relative to the rest of the body.

The term “unit dosage form,” as used herein, refers to physically discrete units suitable as unitary dosages for human and animal subjects, each unit containing a predetermined quantity of peptides of the present invention calculated in an amount sufficient to produce the desired effect in association with a pharmaceutically acceptable diluent, carrier or vehicle. The specifications for the novel unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the peptides will generally be administered in dosages of 0.01 mg to 500 mg V/kg body weight per day, e.g. about 20 mg/day for an average person. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus for example oral dosages in the rat may be ten times the injection dose. A typical dosage may be one injection daily.

Those of skill will readily appreciate that dose levels can vary as a function of the specific compound, the severity of the symptoms and the susceptibility of the subject to side effects. Some of the specific peptides are more potent than others. Preferred dosages for a given complex are readily determinable by those of skill in the art by a variety of means. A preferred means is to measure the physiological potency of a given compound.

Non-Antigen-Specific Immunomodulatory Agents

In certain embodiments of the invention, non-antigen-specific immunomodulatory agents are co-administered with the mevalonate pathway inhibitor (e.g., statin). Non-antigen-specific immunomodulatory agents can be, for example, nucleic acids (e.g., DNA or RNA), such as, e.g., immune modulatory oligonucleotides or polynucleotide vectors encoding an immunomodulatory polypeptide. Also, the non-antigen specific agent can be, e.g., a small organic molecule having immunosuppressive properties. In other embodiments, the non-antigen-specific immunomodulatory agent can be, e.g., a polypeptide. Immunomodulatory polypeptides can include, for example, cytokines, chemokines, interleukins, interferons (e.g., IFN-β), or costimulatory molecules (e.g., CTLA-4). In cases where a natural form of the immunomodulatory polypeptide exists as a membrane bound protein, the polypeptide can be modified into a soluble form (e.g., Ig-fusion with the extracellular domain of the polypeptide). Typically, the non-antigen-specific immunomodulatory agent is not a mevalonate pathway inhibitor. For example, where the mevalonate pathway inhibitor is a statin, the non-antigen-specific agent is a non-statin molecule.

In one preferred embodiment of the invention, the non-antigen-specific immunomodulatory agent is osteopontin or a self-vector comprising a polynucleotide encoding osteopontin. Osteopontin is a pleiotrophic molecule recently identified to play pathogenic roles in autoimmune disease. Treatment with the self-protein osteopontin or with DNA encoding osteopontin induces an anti-osteopontin immunoglobulin response in the host that inhibits the detrimental impact of osteopontin in perpetuating the disease.

Immune Modulatory Sequences. In a preferred embodiment, the non-antigen-specific immunomodulatory protein is an oligonucleotide comprising an immune modulatory sequence.

In one aspect, the immune modulatory sequences of the invention are synthesized oligonucleotides comprised of the following primary structure:

5′-purine-pyrimidine-[X]-[Y]-pyrimidine-pyrimidine-3′ or

5′-purine-purine-[X]-[Y]-pyrimidine-pyrimidine-3′;

wherein X and Y are any naturally occurring or synthetic nucleotide, except that X and Y cannot be cytosine-guanine.

The core hexamer of IMSs can be flanked 5′ and/or 3′ by any composition or number of nucleotides or nucleosides. Preferably, IMSs range between 6 and 100 base pairs in length, and most preferably 16-50 base pairs in length. IMSs can also be delivered as part of larger pieces of DNA, ranging from 100 to 100,000 base pairs. IMSs can be incorporated in, or already occur in, DNA plasmids, viral vectors and genomic DNA. Most preferably IMSs can also range from 6 (no flanking sequences) to 10,000 base pairs, or larger, in size. Sequences present which flank the hexamer core can be constructed to substantially match flanking sequences present in any known immunoinhibitory sequences (IIS). For example, the flanking sequences TGACTGTG-Pu-Pu-X-Y-Pyr-Pyr-AGAGATGA, where TGACTGTG (SEQ ID NO: 76) and AGAGATGA (SEQ ID NO: 77) are flanking sequences. Another preferred flanking sequence incorporates a series of pyrimidines (C, T, and U), either as an individual pyrimidine repeated two or more times, or a mixture of different pyrimidines two or more in length. Different flanking sequences have been used in testing inhibitory modulatory sequences. Further examples of flanking sequences for inhibitory oligonucleotides are contained in the following references: U.S. Pat. Nos. 6,225,292 and 6,339,068 Zeuner et al., Arthritis and Rheumatism, 46:2219-24, 2002.

Particular IMSs of the invention include oligonucleotides containing the following hexamer sequences:

1. 5′-purine-pyrimidine-[X]-[Y]-pyrimidine-pyrimidine-3′ IMSs containing GG dinucleotide cores: GTGGTT (SEQ ID NO: 12), ATGGTT (SEQ ID NO: 13), GCGGTT (SEQ ID NO: 14), ACGGTT (SEQ ID NO: 15), GTGGCT (SEQ ID NO: 16), ATGGCT (SEQ ID NO: 17), GCGGCT (SEQ ID NO: 18), ACGGCT (SEQ ID NO: 19), GTGGTC, (SEQ ID NO: 20) ATGGTC (SEQ ID NO: 21), GCGGTC (SEQ ID NO: 22), ACGGTC (SEQ ID NO: 23), and so forth.

2. 5′-purine-pyrimidine-[X]-[Y]-pyrimidine-pyrimidine-3′ IMSs containing GC dinucleotides cores: GTGCTT (SEQ ID NO: 24), ATGCTT (SEQ ID NO: 25), GCGCTT (SEQ ID NO: 26), ACGCTT (SEQ ID NO: 27), GTGCCT (SEQ ID NO: 28), ATGCCT (SEQ ID NO: 29), GCGCCT (SEQ ID NO: 30), ACGCCT (SEQ ID NO: 31), GTGCTC (SEQ ID NO: 32), ATGCTC (SEQ ID NO: 33), GCGCTC (SEQ ID NO: 34), ACGCTC (SEQ ID NO: 35), and so forth.

3. Guanine and inosine substitutes for adenine and/or uridine substitutes for cytosine or thymine and those substitutions can be made as set forth based on the guidelines above.

A previously disclosed immune inhibitory sequence or IIS, was shown to inhibit immunostimulatory sequences (ISS) activity containing a core dinucleotide, CpG. U.S. Pat. No. 6,225,292. This IIS, in the absence of an ISS, was shown for the first time by this invention to prevent and treat autoimmune disease either alone or in combination with DNA polynucleotide therapy. This IIS contained the core hexamer AAGGTT (SEQ ID NO: 36). That sequence is referred to herein as an immune modulatory sequence or IMS. Other related IISs with a similar motif included within the IMSs of this invention are:

1. 5′-purine-purine-[X]-[Y]-pyrimidine-pyrimidine-3′ IMSs containing GG dinucleotide cores: GGGGTT (SEQ ID NO: 37), AGGGTT (SEQ ID NO: 38), GAGGTT (SEQ ID NO: 39), AAGGTT (SEQ ID NO: 40), GGGGCT (SEQ ID NO: 41), AGGGCT (SEQ ID NO: 42), GAGGCT (SEQ ID NO: 43), AAGGCT (SEQ ID NO: 44), GGGGTC (SEQ ID NO: 45), AGGGTC (SEQ ID NO: 46), GAGGTC (SEQ ID NO: 47), AAGGTC (SEQ ID NO: 48), and so forth.

2. 5′-purine-purine-[X]-[Y]-pyrimidine-pyrimidine-3′ IMSs containing GC dinucleotide cores: GGGCTT (SEQ ID NO: 48), AGGCTT (SEQ ID NO: 49), GAGCTT (SEQ ID NO: 50), AAGCTT (SEQ ID NO: 51), GGGCCT (SEQ ID NO: 52), AGGCCT (SEQ ID NO: 53), GAGCCT (SEQ ID NO: 54), AAGCCT (SEQ ID NO: 55), GGGCTC (SEQ ID NO: 56), AGGCTC (SEQ ID NO: 57), GAGCTC (SEQ ID NO: 58), AAGCTC (SEQ ID NO: 59), and so forth.

3. Guanine and inosine substitutions for adenine and/or uridine substitutions for cytosine or thymine can be made as set forth based on the guidelines above.

Oligonucleotides can be obtained from existing nucleic acid sources, including genomic DNA, plasmid DNA, viral DNA and cDNA, but are preferably synthetic oligonucleotides produced by oligonucleotide synthesis. IMS can be part of single-strand or double-stranded DNA, RNA and/or oligonucleosides.

IMSs are preferentially oligonucleotides that contain unmethylated GpG oligonucleotides. Alternative embodiments include IMSs in which one or more adenine or cytosine residues are methylated. In eukaryotic cells, typically cytosine and adenine residues can be methylated.

IMSs can be stabilized and/or unstabilized oligonucleotides. Stabilized oligonucleotides mean oligonucleotides that are relatively resistant to in vivo degradation by exonucleases, endonucleases and other degradation pathways. Preferred stabilized oligonucleotides have modified phophate backbones, and most preferred oligonucleotides have phophorothioate modified phosphate backbones in which at least one of the phosphate oxygens is replaced by sulfur. Backbone phosphate group modifications, including methylphosphonate, phosphorothioate, phophoroamidate and phosphorodithionate internucleotide linkages, can provide antimicrobial properties on IMSs. The IMSs are preferably stabilized oligonucleotides, preferentially using phosphorothioate stabilized oligonucleotides.

Alternative stabilized oligonucleotides include: alkylphosphotriesters and phosphodiesters, in which the charged oxygen is alkylated; arylphosphonates and alkylphosphonates, which are nonionic DNA analogs in which the charged phosphonate oxygen is replaced by an aryl or alkyl group; or/and oligonucleotides containing hexaethyleneglycol or tetraethyleneglycol, or another diol, at either or both termini. Alternative steric configurations can be used to attach sugar moieties to nucleoside bases in IMSs.

The nucleotide bases of the IMS which flank the modulating dinucleotides may be the known naturally occurring bases or synthetic non-natural bases. Oligonucleosides may be incorporated into the internal region and/or termini of the IMS-ON using conventional techniques for use as attachment points, that is as a means of attaching or linking other molecules, for other compounds, including self-lipids, self-polypeptides, self-glycoproteins, self-glycolipids, self-carbohydrates, and posttranslationally-modified self-polypeptides or self-glycoproteins, or as attachment points for additional immune modulatory therapeutics. The base(s), sugar moiety, phosphate groups and termini of the IMS-ON may also be modified in any manner known to those of ordinary skill in the art to construct an IMS-ON having properties desired in addition to the modulatory activity of the IMS-ON. For example, sugar moieties may be attached to nucleotide bases of IMS-ON in any steric configuration.

The techniques for making these phosphate group modifications to oligonucleotides are known in the art and do not require detailed explanation. For review of one such useful technique, the intermediate phosphate triester for the target oligonucleotide product is prepared and oxidized to the naturally occurring phosphate triester with aqueous iodine or with other agents, such as anhydrous amines. The resulting oligonucleotide phosphoramidates can be treated with sulfur to yield phophorothioates. The same general technique (excepting the sulfur treatment step) can be applied to yield methylphosphoamidites from methylphosphonates. For more details concerning phosphate group modification techniques, those of ordinary skill in the art may wish to consult U.S. Pat. Nos. 4,425,732; 4,458,066; 5,218,103 and 5,453,496, as well as Tetrahedron Lett. at 21:4149 25 (1995), 7:5575 (1986), 25:1437 (1984) and Journal Am. Chem Soc., 93:6657 (1987), the disclosures of which are incorporated herein for the purpose of illustrating the level of knowledge in the art concerning the composition and preparation of IMSs.

A particularly useful phosphate group modification is the conversion to the phosphorothioate or phosphorodithioate forms of the IMS-ON oligonucleotides. Phosphorothioates and phosphorodithioates are more resistant to degradation in vivo than their unmodified oligonucleotide counterparts, making the IMS-ON of the invention more available to the host.

IMS-ON can be synthesized using techniques and nucleic acid synthesis equipment which are well-known in the art. For reference in this regard, see, e.g., Ausubel, et al., Current Protocols in Molecular Biology, Chs. 2 and 4 (Wiley Interscience, 1989); Maniatis, et al., Molecular Cloning: A Laboratory Manual (Cold Spring Harbor Lab., New York, 1982); U.S. Pat. No. 4,458,066 and U.S. Pat. No. 4,650,675. These references are incorporated herein by reference for the purpose of demonstrating the level of knowledge in the art concerning production of synthetic oligonucleotides.

Alternatively, IMS-ON can be obtained by mutation of isolated microbial ISS-ODN to substitute a competing dinucleotide for the naturally occurring CpG motif and the flanking nucleotides. Screening procedures which rely on nucleic acid hybridization make it possible to isolate any polynucleotide sequence from any organism, provided the appropriate probe or antibody is available. Oligonucleotide probes, which correspond to a part of the sequence encoding the protein in question, can be synthesized chemically. This requires that short, oligo-peptide stretches of amino acid sequence must be known. The DNA sequence encoding the protein can also be deduced from the genetic code, however, the degeneracy of the code must be taken into account.

For example, a cDNA library believed to contain an ISS-containing polynucleotide can be screened by injecting various mRNA derived from cDNAs into oocytes, allowing sufficient time for expression of the cDNA gene products to occur, and testing for the presence of the desired cDNA expression product, for example, by using antibody specific for a peptide encoded by the polynucleotide of interest or by using probes for the repeat motifs and a tissue expression pattern characteristic of a peptide encoded by the polynucelotide of interest. Alternatively, a cDNA library can be screened indirectly for expression of peptides of interest having at least one epitope using antibodies specific for the peptides. Such antibodies can be either polyclonally or monoclonally derived and used to detect expression product indicative of the presence of cDNA of interest.

Once the ISS-containing polynucleotide has been obtained, it can be shortened to the desired length by, for example, enzymatic digestion using conventional techniques. The CpG motif in the ISS-ODN oligonucleotide product is then mutated to substitute an “inhibiting” dinucleotide—identified using the methods of this invention—for the CpG motif. Techniques for making substitution mutations at particular sites in DNA having a known sequence are well known, for example M13 primer mutagenesis through PCR. Because the IMS is non-coding, there is no concern about maintaining an open reading frame in making the substitution mutation. However, for in vivo use, the polynucleotide starting material, ISS-ODN oligonucleotide intermediate or IMS mutation product should be rendered substantially pure (i.e., as free of naturally occurring contaminants and LPS as is possible using available techniques known to and chosen by one of ordinary skill in the art).

The IMS of the invention may be used alone or may be incorporated in cis or in trans into a recombinant self-vector (plasmid, cosmid, virus or retrovirus) which may in turn code for any self-polypeptide deliverable by a recombinant expression vector. For the sake of convenience, the IMSs are preferably administered without incorporation into an expression vector. However, if incorporation into an expression vector is desired, such incorporation may be accomplished using conventional techniques as known to one of ordinary skill in the art. For review those of ordinary skill would consult Ausubel, Current Protocols in Molecular Biology, supra.

Briefly, construction of recombinant expression vectors employs standard ligation techniques. For analysis to confirm correct sequences in vectors constructed, the ligation mixtures may be used to transform a host cell and successful transformants selected by antibiotic resistance where appropriate. Vectors from the transformants are prepared, analyzed by restriction and/or sequenced by, for example, the method of Messing, et al., (Nucleic Acids Res., 9:309, 1981), the method of Maxam, et al., (Methods in Enzymology, 65:499, 1980), or other suitable methods which will be known to those skilled in the art. Size separation of cleaved fragments is performed using conventional gel electrophoresis as described, for example, by Maniatis, et al., (Molecular Cloning, pp. 133-134, 1982).

Host cells may be transformed with the expression vectors of this invention and cultured in conventional nutrient media modified as is appropriate for inducing promoters, selecting transformants or amplifying genes. The culture conditions, such as temperature, pH and the like are those previously used with the host cell selected for expression, and will be apparent to the ordinarily skilled artisan.

If a recombinant expression vector is utilized as a carrier for the IMS-ON of the invention, plasmids and cosmids are particularly preferred for their lack of pathogenicity. However, plasmids and cosmids are subject to degradation in vivo more quickly than viruses and therefore may not deliver an adequate dosage of IMS-ON to prevent or treat an inflammatory or autoimmune disease.

Most of the techniques used to construct vectors, and transfect and infect cells, are widely practiced in the art, and most practitioners are familiar with the standard resource materials that describe specific conditions and procedures.

Co-Administration of Agents for Treatment of Autoimmune Disease:

As noted above, the agents of the present invention (mevalonate inhibitor and a second immunomodulatory agent) are co-administered to a subject for the treatment of autoimmune disease. Co-administration means administration to a subject of the agents, each in an effective dose, such that the agents are present and active in the subject at the same time. Thus, co-administration refers to administration to the same subject and not necessarily to the same site or by the same route of administration. In some embodiments, the agents are administered at the same time. In other embodiments, the agents are coordinately administered so that the first agent is present and active in the subject before the second agent is administered, with both agents present and active following administration of the second agent. The agents are typically co-administered coordinately as separate formulations. Further, the agents can be administered by the same routes of administration or by different routes. Different routes of administration can include, for example, systemic and local routes for the agents within the same treatment regimen. For example, where combination therapy includes co-administration of a statin and a self-vector encoding a self-polypeptide, the statin can be delivered orally while the self-vector can be administered intramuscularly.

In each of the embodiments of the invention described herein, the agents are delivered in a manner consistent with conventional methodologies associated with the management of the autoimmune disorder for which treatment or prevention is sought. In accordance with the disclosure herein, an effective regime of the agents is administered to a subject in need of such treatment for a time and under conditions sufficient to treat the autoimmune reactions.

Subjects for the combination therapy according to the invention include patients at high risk for developing an autoimmune disease as well as patients presenting with existing autoimmune disease. Typically, the subject has been diagnosed as having an autoimmune disease for which treatment is sought. Further, subjects can be monitored during the course of the treatment for any change in autoimmune disease symptoms in response to the treatment.

To identify subject patients for treatment according to the methods of the invention, accepted screening methods are employed to determine risk factors associated with specific autoimmune disorders or to determine the status of an existing disorder identified in a subject. Such methods can include, for example, determining whether an individual has relatives who have been diagnosed with an autoimmune disease. Screening methods can also include, for example, conventional work-ups to determine familial status for a particular autoimmune or inflammatory disease known to have a heritable component. Toward this end, nucleotide probes can be routinely employed to identify individuals carrying genetic markers associated with a particular autoimmune disease of interest. In addition, a wide variety of immunological methods are known in the art that are useful to identify markers for specific autoimmune diseases. For example, various ELISA immunoassay methods are available and well-known in the art that employ monoclonal antibody probes to detect autoantibodies associated with specific physiological markers of autoimmune disease. Such screening may be implemented as indicated by known patient symptomology, age factors, related risk factors, etc. These methods allow the clinician to routinely select patients in need of the methods described herein for treatment of autoimmune disease. In accordance with these methods, the combination therapy may be implemented as an independent prevention or treatment program or as a follow-up, adjunct, or coordinate treatment regimen to other treatments.

The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease, where the disease has recurring symptoms (i.e. is multiphasic). The presymptomatic, or preclinical stage will be defined as that period when there is T cell involvement at the site of disease, e.g. central nervous system, etc., but the loss of function is not yet severe enough to produce the clinical symptoms indicative of overt disease. T cell involvement may be evidenced by the presence of elevated numbers of T cells at the site of disease, the presence of T cells specific for autoantigens, the release of performs and granzymes at the site of disease, response to immunosuppressive therapy, etc.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

Determining the effectiveness of a regimen may utilize assays directed to determination of T cell responses. The assay may determine the level of reactivity, e.g. based on the number of reactive T cells found in a sample, as compared to a negative control from a naive host, or standardized to a data curve obtained from one or more patients. In addition to detecting the qualitative and quantitative presence of auto-antigen reactive T cells, the T cells may be typed as to the expression of cytokines known to increase or suppress inflammatory responses. It may also be desirable to type the epitopic specificity of the reactive T cells.

T cells may be isolated from patient peripheral blood, lymph nodes, or preferably from the site inflammation. Reactivity assays may be performed on primary T cells, or the cells may be fused to generate hybridomas. Such reactive T cells may also be used for further analysis of disease progression, by monitoring their in situ location, T cell receptor utilization, etc. Assays for monitoring T cell responsiveness are known in the art, and include proliferation assays and cytokine release assays.

Proliferation assays measure the level of T cell proliferation in response to a specific antigen, and are widely used in the art. In an exemplary assay, patient lymph node, blood or spleen cells are obtained. A suspension of from about 10⁴ to 10⁷ cells, usually from about 10⁵ to 10⁶ cells is prepared and washed, then cultured in the presence of a control antigen, and test antigens. The test antigens may be peptides of any autologous antigens suspected of inducing an inflammatory T cell response. The cells are usually cultured for several days. Antigen-induced proliferation is assessed by the monitoring the synthesis of DNA by the cultures, e.g. incorporation of ³H-thymidine during the last 18H of culture.

Enzyme linked immunosorbent assay (ELISA) assays are used to determine the cytokine profile of reactive T cells, and may be used to monitor for the expression of such cytokines as IL-2, IL-4, IL-5, IL-10, γ-IFN, etc. The capture antibodies may be any antibody specific for a cytokine of interest, where supernatants from the T cell proliferation assays, as described above, are conveniently used as a source of antigen. After blocking and washing, labeled detector antibodies are added, and the concentrations of protein present determined as a function of the label that is bound.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect in the absence of T cell mediated inflammation.

It is to be understood that this invention is not limited to the particular methodology, protocols, formulations and reagents described, as such may, of course, vary. It is also to be understood that the terminology used herein is for the purpose of describing particular embodiments only, and is not intended to limit the scope of the present invention which will be limited only by the appended claims.

All publications mentioned herein are incorporated herein by reference in their entirety for all purposes, including the purpose of describing and disclosing, for example, the methods and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, and pressure is at or near atmospheric.

### EXAMPLE 1

### Atorvostatin for Treatment of an Animal Model for Multiple Sclerosis

It was examined whether atorvastatin (Lipitor®) could inhibit the proinflammatory response in experimental autoimmune encephalomyelitis (EAE), a Th1 mediated central nervous system (CNS) demyelinating disease that serves as a model for multiple sclerosis (MS). Daily oral administration of atorvastatin initiated at the onset of MOG p35-55-induced chronic EAE in C57BL/6 mice reversed paralysis. Atorvastatin also ameliorated the relapses in SJL/J mice when given after the acute attack in relapsing remitting EAE induced by PLP p139-151. Acute EAE was also prevented in MBPAc1-11 treated Tg mice. Histological evaluation of brains and spinal cords taken from atorvastatin-treated mice, showed significant reduction in both the number of the perivascular lesions as well as the extent of infiltration in those lesions. CNS MHC class II transactivator (CIITA) expression, including expression of individual promoter (p) I, pIII and pIV transcripts, was reduced in atorvastatin-treated mice. Atorvastatin treatment was associated with reduction of CNS-autoantigen-specific proliferative T cell responses, decrease in IFN-γ and IL-2 secretion and increase of IL-4, and IL-10 secretion by these T cells. Thus, atorvastatin treatment promoted a Th2 bias. These results demonstrate that atorvastatin is an effective immunomodulatory agent for the treatment of demyelinating disease.

Methods:

Experimental Procedures

Animals. Female SJL/J, B10.PL and C57BL/6 mice (8 to 12-week-old) were purchased from the Jackson Laboratory (Bar Harbor, Me.). MBP Ac 1-11 transgenic (tg) TCR mice were backcrossed with B10.PL mice to obtain susceptibility to EAE. All animal protocols were approved by the Division of comparative Medicine at Stanford and in accordance with the National Institutes of Health guidelines.

Peptides. Peptides were synthesized on a peptide synthesizer (model 9050; MilliGen, Burlington, Mass.) by standard 9-fluorenylmethoxycarbonyl chemistry. Peptides were purified by HPLC. Structures were confirmed by amino acid analysis and mass spectroscopy. Peptides used in these experiments were mouse MBPAc 1-11 l (Ac-ASQKRPSQRHG) (SEQ ID NO: 60), MOG35-55 (MEVGWYRSPFSRVVHLYRNGK) (SEQ ID NO: 61), PLP139-151 (HCLGKWLGHPDKF) (SEQ ID NO: 62); and HSVP 16 (DMTPADALDDRDLEM) (SEQ ID NO: 63)—a viral peptide used as a negative control in the proliferation and cytokine assays.

Drug Treatments. Atorvastatin (Lipitor®) tablets were obtained commercially and dissolved in PBS. Mice were subjected to oral administration of 0.5 ML Atorvastatin solution (1 or 10 mg/kg) or only PBS once daily using 18 mm feeding needles. The periods of the atorvastatin treatment are indicated in the result section.

EAE Induction. Relapsing remitting EAE was induced in SJL/J mice with 100 μg of PLP139-151 peptide, chronic progressive EAE was induced either in C57BL/6 or MBP Ac1-11 TCR Tg mice with 100 μg of MOG35-55 peptide or 100 μg of MBP Ac1-11 peptide, respectively. All peptides were dissolved in PBS at a concentration of 2 mg/ml and emulsified with an equal volume of CFA, which consists of incomplete Freund's adjuvant supplemented with 4 mg/ml heat-killed mycobacterium tuberculosis H37Ra (Difco Laboratories, Detroit, Mich.). Mice were injected subcutaneously with 0.1 ml of the peptide emulsion. On the day of peptide immunization and 48 hr later, only C57BL/6 mice and MBP Ac 1-11 TCR Tg mice were also injected intravenously with 0.1 ml of 1 μg/ml Bordetella pertussis toxin in PBS. Mice were clinically scored as follows: 0, no paralysis; 1, tail weakness or paralysis; 2, hindlimb weakness or paralysis; 3, hindlimb paralysis and forelimb weakness; 4, hindlimb and forelimb paralysis; and 5, moribund or death.

Ag specific ex-vivo T cell proliferation assay. Atorvastatin 1 mg/kg or 10 mg/kg or PBS daily treatments started 2 days before EAE induction in all the different strains. 10 days after EAE induction (i.e. 12 days after Atorvastatin treatments) draining lymph nodes and spleens were removed from control, 1 mg/kg or 10 mg/kg Atorvastatin treated SJL/J, C57BL/6 and MBP Ac1-11 transgenic mice. Lymph node cells (LNCs) or splenocytes were cultured in vitro for specific proliferative response to the specific encephalogenic peptide (PLP 139-151, MOG 35-55 or MBP Ac1-11, respectively). LNCs were prepared in 96-well microtiter plates in a volume of 0.2 ml/well at a concentration of 5×10⁶ cells/ml. The culture medium consisted of enriched RPMI (RPMI 1640 supplemented with L-glutamine [2 mM], sodium pyruvate [1 mM], nonessential amino acids (0.1 mM], penicillin [100 U/ml], streptomycin [0.1 mg/ml], 2-ME (5×10⁻⁵ M]) supplemented with 1% autologous fresh normal mouse serum with the addition of different peptides concentrations. Cultures were incubated in 37° C. in humidified air containing 5% CO₂. Cultures taken from SJL/J or C57BL/6 mice were incubated for 72 h whereas cultures from MBPAc-1-11 Tg mice were incubated for 48 hours and then were pulsed for 18 hr with 1 μCi/well of [³H] thymidine. The cells were then harvested and counted in a β counter.

Cytokine Profile Determination. Lymph node cells and spleen cells from EAE donors were stimulated in vitro (2.5×10⁶ cells/ml) in 24-well plates with or without the encephalogenic peptide or with CoA as positive control. Cell culture supernatants were collected at different time points for measurements of cytokine levels: 48 hours for IL-2, 72 hours for IFN-γ and TNF α, and 120 hours for IL-4 and IL-10. Cytokine levels were determined using specific ELISA kits for the corresponding cytokines according to the manufacturers protocols (PharMingen, San Diego, Calif., USA).

Total RNA Isolation. Mice were sacrificed and perfused with 20 ml of cold sterile PBS. Brains were immediately isolated and total RNA was isolated using Trizol reagent (Invitrogen) as recommended in the manufacturer protocol. The amounts of the total RNA were then measured at 260 nm.

Evaluation of CIITA promoter-specific mRNA expression by real-time (kinetic) RT-PCR. One step RT-PCR is performed as described in Baranzini et al. (2000) J. Immunol 165:6576. A master mix is prepared with 400 μM dUTP and 200 μM each of dATP, dCTP, and dGTP; 0.2 μM each oligonucleotide primer, 0.2×SYBR green in DMSO (1% final concentration); 2.5% glycerol; 1U uracyl N-glycosilase; 4 mM Mn (OAc)₂ and 5U rTth polymerase. RT-PCR parameters: initial incubation 10 min at 45° C. with activating uracyl N-glycosilase followed by RT 30 min at 60° C.; 50 cycles at 95° C. for 15 s and 57° C. for 30 s. β-actin is amplified from all samples as a housekeeping gene to normalize expression. A control without template is included for each primer set. For quantification, a 10-fold dilution series of a CIITA run-off transcript (10⁷ to 10² initial CIITA copies) is included in each reaction plate. Data are analyzed by software Sequence Detection Systems program and transferred to an MS Excel spread sheet for analysis. A calibration curve is generated by plotting CIITA (run-off transcript) for each 10-fold dilution against the number of cycles required for each product to exceed a preset threshold (Ct). Ct values are compared to those obtained on a standard curve. Primers for common CIITA (nt 2374-2458): 5′-GCCCACGAGACACAGCAA (SEQ ID NO: 64) and 5′-TGAGCCGGGTGCCCAGGAA (SEQ ID NO: 65). 5′ (forward) promoter-specific primers: pI CIITA (pl nt 259) 5′-CCTGACCCTGCTGGAGAA (SEQ ID NO: 66); pIII CIITA (pIII nt 112): 5′-GCATCACTCTGCTCTCTAA (SEQ ID NO: 67); pIV CIITA: (pIV nt 43): 5′-TGCAGGCAGCACTCAGM (SEQ ID NO: 68). CIITA (nt 265) reverse primer for promoter-specific transcripts: 5′-GGGGTCGGCACTGTTAA (SEQ ID NO: 69). β-actin: (301-538): 5′-CGACCTGGGGATCTTCTA (SEQ ID NO: 70) and 5′-TCGTGCCCTCAGCTTCCAA (SEQ ID NO: 71).

Western Blot analysis for STAT-6 and STAT-4 phosphorylation. Western Blot analysis was performed as described in Garren et al. (2001) Immunity 15:15, with minor modifications. Lymph nodes from control and atorvastatin-treated mice were homogenized in T-PER protein extraction buffer (Pierce,), with 20 μg/ml aprotinin, 20 μg/ml leupeptin, 1.6 mM Pefablock SC (Roche), 10 mM NaF, 1 mM Na₃VO₄ and 1 mM Na₄P₂O₇ (Sigma, St. Louis, Mo.). All procedures were handled on ice. As a positive control, lymph node cells from naive mice were isolated and cultured for one hour with mouse recombinant IL-4 (10 ng/ml) or INF-γ (100 units/ml), for STAT6 and STAT4 expression respectively. Protein concentrations were determined by BCA protein assay (Pierce). Lysate was added to 3×SDS loading buffer (Cell Signaling Technology) with 40 mM DTT. Products were separated by electrophoresis on a 4-15% SDS-PAGE gradient gel (BioRad). Pre-stained markers (Invitrogen) were used to determine MW. Gels were blotted to PVDF membranes at 100 V in 25 mM Tris, 192 mM glycine and 20% (v/v) methanol, then blocked 1 hr at RT with Tris-buffered saline (TBS) containing 0.1% Tween-20 and 5% nonfat dry milk. After washing in TBS and 0.1% Tween 20, membranes were hybridized overnight at 4° C. with anti-phospho-STAT6 Antibody or anti-phospho-STAT4 antibody (Zymed, South San Francisco, Calif.) diluted 1:1000 in TBS, 0.1% Tween 20 and 5% BSA, the membranes were then processed by ECL Plus prebetween protocol (Amersham Life Sciences) for visualization of the bands by chemiluminescence. Membranes were stripped in 100 mM 2-mercaptoethanol, 2% (w/v) SDS and 62.5 mM Tris (pH 7.4) for 30 min at 60° C., then probed with anti-CD3ζ (Pharmingen, San Diego, Calif.) or anti Stat6 or anti Stat4 (both obtained from Santa Cruz Biotechnology, Santa Cruz, Calif.) as a control to verify equal loading amounts.

Histopathology. Mice were sacrificed and perfused with 20 ml cold PBS followed by 20 ml of cold 4% paraformaldehyde. Brain and spinal cord were isolated and subjected to paraffin embedding procedure; sections were then subjected to hematoxylin and eosin-staining. Histological examination was performed on 10 sections of each mouse, and each section was evaluated on histological score without knowledge of the treatment status of the animal.

Statistical analysis. Data are presented as mean±SE. Significance of difference between two groups was examined using the Student t test. A value of p<0.05 was considered significant. One-way multiple range ANOVA test with significance level of p<0.05 was performed for multiple compression as well.

Results.

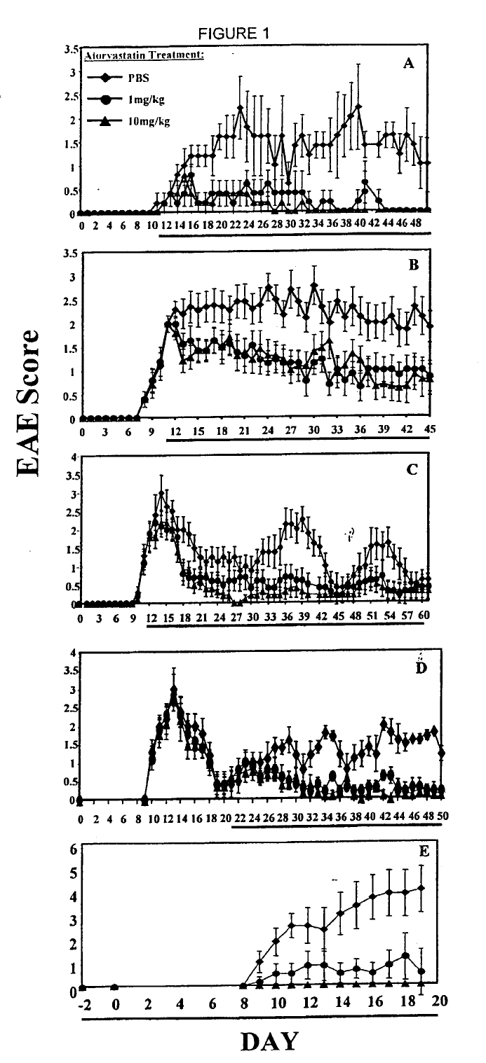

Atorvastatin reverses and prevents an on-going chronic relapsing EAE or chronic progressive EAE in mice. Initially, atorvastatin was tested for prevention of chronic EAE in C57B1/6 female mice induced by immunization with the immunodominant determinant of myelin oligodendrocyte glycoprotein (MOG), p35-55. As shown in FIG. 1A, daily oral treatment starting at the time of EAE onset with either 1 mg/kg (approximately equivalent to the highest approved adult dose of 80 mg) or 10 mg/kg atorvastatin suppressed EAE induction. Treatment after onset also ameliorated EAE (FIG. 1B). Atorvastatin treatment was tested in chronic relapsing EAE in SJL/J mice induced by immunization with encephalitogenic proteolipoprotein (PLP) peptide, p139-151. Not only was atorvastatin effective in prevention of relapsing EAE (FIG. 1C), but there was also reversal of ongoing relapsing EAE when treatment was begun after recovery from acute EAE (FIG. 1D). Atorvastatin successfully prevented acute EAE progression in MBP Ad-11 Tg mice induced by immunization with encephalitogenic myelin basic protein (MBP) peptide, pAd-li (Figure IE).

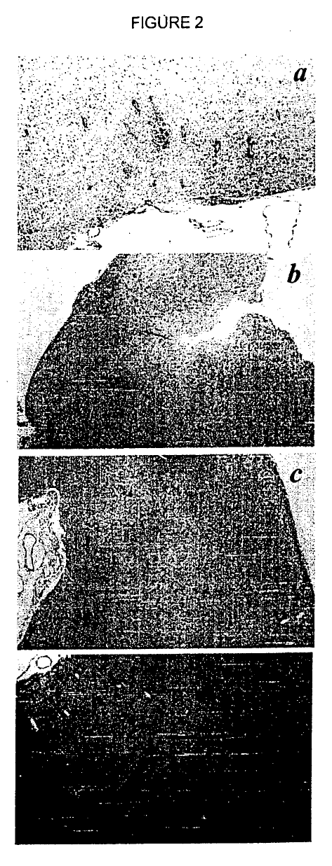

Mice from each group of all five experiments were sacrificed and brains and spinal cords were taken for CNS histological evaluation. FIG. 2 shows a representative H and E staining of brains taken from experiment A (see FIG. 1A) at day 11 after atorvastatin treatment has begun, thus 22 days after EAE induction in C57BL/6 mice. H&E sagittal brain sections taken from PBS treated C57BL/6 mice (a), from 1 mg/kg treatment (b), from 10 mg/kg treatment (c) and from naive C57BL/6 as negative control (d). Sections are representative sections from 2 mice of each group.

Hematoxylin and easin staining revealed a reduction in number and size of CNS infiltrates in atorvastatin-treated mice (shown in FIG. 2B and FIG. 2C), in comparison to PBS treated mice and naive mice (FIGS. 2A and 2D, respectively). Thus, inhibition and prevention of disease manifestation by atorvastatin oral treatments was confirmed, and demonstrated histologically at the site of inflammation (CNS). Spinal cords and brains from representative members from the other 4 animal experiments were subjected to the same analysis, and similar results obtained.

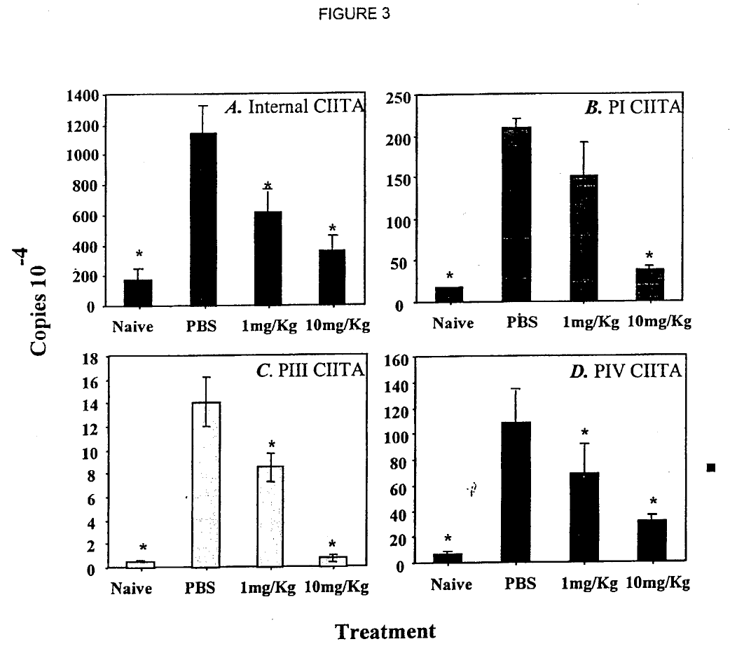

Atorvastatin downregulates CIITA expression at the site of inflammation (CNS) during EAE. In the normal central nervous system (CNS), expression of MHC class II is minimal although it is found to be highly up-regulated on microglia cells in EAE induced in mice. This expression is regulated by the factor class II transactivator (CIITA), which is required for activation of MHC class II genes especially CIITA pVI that regulate the expression in microglia cells (the major antigen presenting cells in the CNS). It was also reported that atorvastatin could inhibit the expression of MHC II, through the effect on the CIITA p IV gene. Since the histological results pointed out a reduction of infiltrates to the brains of the atorvastatin treated mice comparing with the control EAE mice, it was explored whether atorvastatin inhibits the CIITA expression in vivo in the site of inflammation. 3 groups of SJL/J mice were treated orally with either 1 mg/kg atorvastatin, 10 mg/kg or with PBS only (control). A fourth group of naive mice were added as a negative control of CIITA expression. The 3 treated groups were subjected to a daily treatments started 2 days before induction of EAE. On day 10 after the induction (12 days of the different treatments) mice from all 4 groups were sacrificed and perfused with 20 ml of cold PBS. Brains were isolated and subjected to total RNA preparation as described in the method section. RNA was subjected to real time PCR (RT-PCR) to measure the effect of atorvastatin treatments and vehicle-treated EAE on the expression of Promoter-specific CIITA transcripts at site of inflammation (CNS) in vivo. Results are demonstrated in FIG. 3.

Atorvastatin treatment inhibited the total expression of CIITA transcripts in a dose response matter (FIG. 3A). Specific CIITA analysis showed that atorvastatin treatment inhibits all three specific isoforms of CIITA (FIGS. 3B, 3C and 3D). Interestingly, atorvastatin showed a dose dependent inhibition of CIITA Ply transcript (known to be specific for regulation of MHC II expression on microglia in ONS) but also unexpectedly, PI and PIII transcripts as well, which are known to be specific for dendntic cells and B cells, respectively. The P1 and Pill transcripts as shown to affected by atorvastatin in vitro.

These results demonstrate the direct inhibition of CIITA isoforms in the brain treated with atorvastatin, which could be a major factor in inhibiting the expression of MHC II in the CNS and thus preventing the massive infiltration of mononuclear cells into the CNS and reversal of EAE.

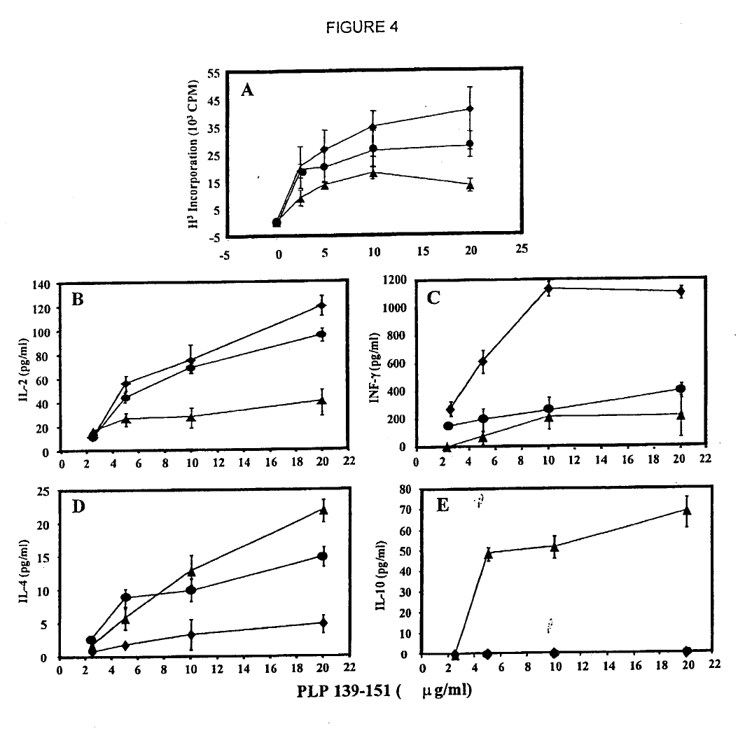

Atovastatin promotes development of a Th2 bias. Lymphocytes isolated from spleens and lymph nodes from SJL/J female mice immunized with PLP p139-151 for EAE induction and treated with either atorvastatin or vehicle (control) were isolated after 10 days of treatment and examined for proliferation and cytokine production. As shown in FIG. 4A, PLP p139-151-specific proliferative responses were suppressed in a dose-related fashion. Production of IL-2, a Th1 cytokine, was reduced, although to a much greater extent in mice treated with 10 mg/kg (FIG. 4B). There was a dramatic reduction in secretion of IFN-γ, the hallmark Th1 cytokine, at both treatment doses (FIG. 4C). IL-4, a key anti-inflammatory cytokine, was induced at both treatment doses (FIG. 4D), while in this experiment secretion of IL-10, another anti-inflammatory Th2 cytokine, was observed at the higher treatment dose (FIG. 4E). Thus, atorvastatin suppressed Th1 cytokine production and promoted Th2 cytokines. These experiments were repeated in C57BL/6 and MBP Ac1-11-specific transgenic. Similar data were obtained. As described above for SJL/J mice, atorvastatin promoted an almost identical Th2 bias in these mice. In these in vitro experiments we have not observed increased cell death.

Atorvastatin causes activation of Stat6. In order to demonstrate that IL-4 production in the atorvastatin cause a bias from Th1 to Th2, we wanted to explore whether functional IL-4 cytokine was actually expressed during atorvastatin treatment. IL-4 is known to act through the IL-4 receptor to specifically activate STAT6, a member of the signal transducers and activators of transcription family thus it's expected to be phosphorylated when an IL-4 dependent Th2 bias occurs. SJL/J mice were daily treated with oral administrations of either atorvastatin (1 mg/kg and 10 mg/kg) or only PBS and EAE was induced, by administrating PLP/CFA, two days after the beginning of the statin treatment.

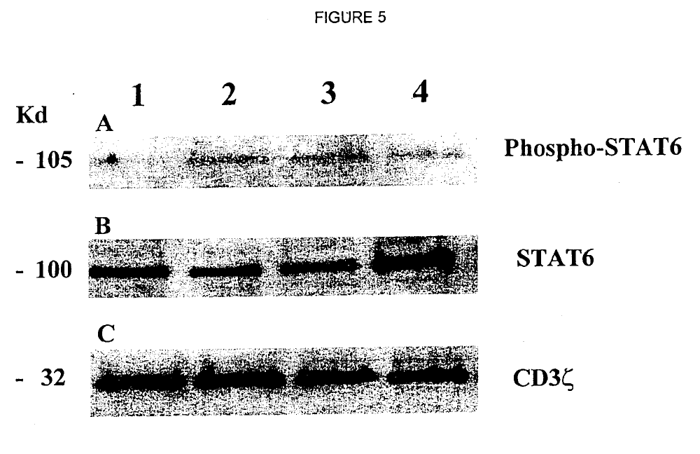

10 days after the EAE induction all groups were sacrificed and draining lymph nodes were dissected. Protein lysates were isolated from the lymph node cells and probed for the presence of activated STAT6 by Western blotting using a polyclonal antibody specific for the phosphorylated form of STAT6. As for positive control, lymph node cells were isolated from naive mice and incubated with mouse recombinant IL-4 (10 ng/ml) for one hour. Protein lysates were extracted in a similar manner. As shown in FIG. 5, phosphorylated STAT6 is seen in lymph nodes from atorvastatin treated mice (lane 2 and 3) and from the positive control (lane 4) whereas, PBS treated mice show no detectable phosphorylation of it (lane 1). The phosphorylated STAT6 identified runs at approximately 100 kDa according to pre stained markers.

### EXAMPLE 2

### Polynucleotide Therapy Comprising Administration of DNA Encoding the Self-Protein PLP for Prevention of an Animal Model of Multiple Sclerosis

PLP self-vector. A polynucleotide encoding an epitope of the PLP self-protein was constructed by annealing two oligonucleotides with a 16 mer overlapping complementary sequence (underlined), and extending with DNA polymerase and dNTPs:

| | |
|---|---|
| PLP (139-151): | |
| 5′-CTCGAGACCATGCATTGTTTGGGAAAATGGCTAGGACATCCCGA | (SEQ ID NO:72) |
| CAAGTTTTCTAGATAGCTA-3′; | |
| PLP (139-151) L144/R147: | |
| 5′-CTCGAGACCATGCATTGTTTGGGAAAACTACTAGGACGCCCCG | (SEQ ID NO:73) |
| ACAAGTTTTCTAGATAGCTA-3′. | |

These oligonucleotide duplexes were designed to incorporate Xho I and Xba I restriction sites. The products were cloned into the multiple cloning region of pTARGET Vector (Promega, Madison, Wis.), a mammalian expression vector driven by the CMV promoter. Positive clones were identified by color screening and correct orientation of the inserts was confirmed by DNA automatic sequencing. Purification of the plasmid DNA was done by Wizard plus Maxipreps (Promega) according to manufacturer instructions were injected with 0.05 ml of plasmid DNA (1 mg/ml in PBS), in the same muscle.

Polynucleotide therapy protocol. Experimental animals were injected in the left quadraceps with 0.1 ml of 0.25% bupivacaine-HCl (Sigma, St. Louis, Mo.) in PBS. Two and ten days later, mice were injected with 0.05 ml of plasmid DNA (1 mg/ml in PBS), in the same muscle.

EAE induction. PLP139-151 peptide was dissolved in PBS to a concentration of 2 mg/ml and emulsified with an equal volume of Incomplete Freund's Adjuvant supplemented with 4 mg/ml heat-killed mycobacterium tuberculosis H37Ra (Difco Laboratories, Detroit, Mich.). Mice were injected subcutaneously with 0.1 ml of the peptide emulsion and, on the same day and 48 h later, intravenously with 0.1 ml of 4 □g/ml Bordetella Pertussis toxin in PBS. Experimental animals were scored as follows: 0=no clinical disease; 1=tail weakness or paralysis; 2=hind limb weakness; 3=hind limb paralysis; 4=forelimb weakness or paralysis; 5=moribund or dead animal.

To determine whether injection of DNA encoding PLP sequences is effective in protecting mice from EAE induction, the PLP139-151 self-vector was injected, intramuscularly, twice, at one week intervals. Ten days after the last injection, mice were challenged with the PLP 139-151 peptide emulsified in CFA. Amelioration of acute clinical disease is observed in the animals treated with the PLP139-151 self-vector, as compared with the control plasmid group. Onset of disease was delayed compared to the control plasmid group (11.5±0.5 days, p<0.008), mean peak disease severity was reduced (p<0.005), and mean disease score was reduced (p<0.0005). In addition, other groups were injected with either a) a self-vector comprising a polynucleotide encoding the altered peptide ligand PLP p139-151 (W144>L, H147>R), b) a self-vector comprising a polynucleotide encoding the PLP epitope p178-191. Onset of disease was delayed (11.6±0.5 days, p<0.009) and mean peak disease score was reduced (p<0.02) with the self-vector encoding the altered self-peptide ligand (W144, H147). Also, onset of disease was delayed (11.5±0.4 days, p<0.003), mean peak disease severity was reduced (p<0.007), and mean disease score was reduced (p<0.0001) with the self-vector comprising the polynucleotide encoding the PLP self-peptide p178-191.

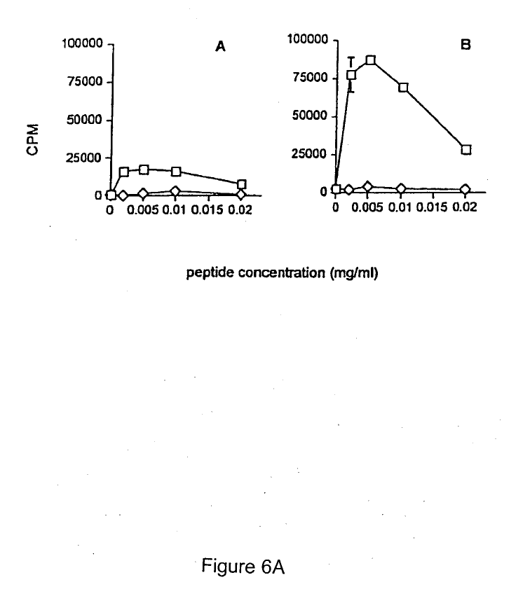

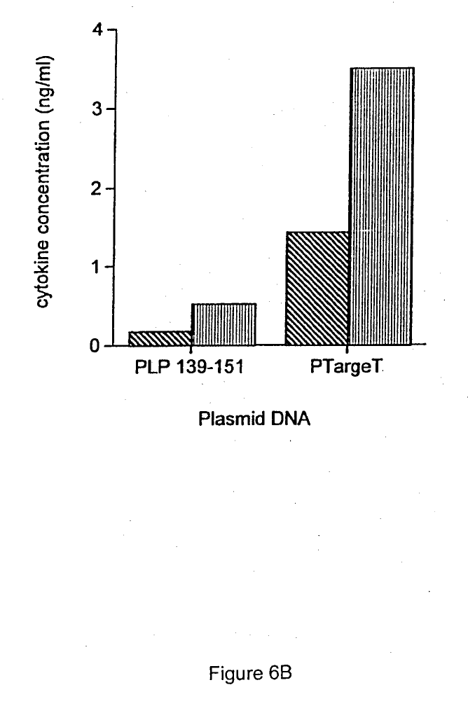

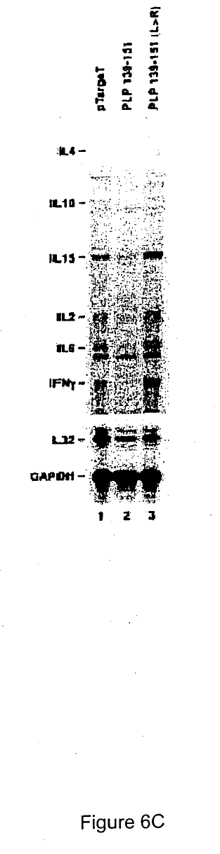

Mice, injected with DNA and further challenged with the encephalitogenic peptide PLP139-151, were sacrificed after resolution of the acute phase of the clinical disease. Draining LNC were restimulated in vitro with the PLP139-151 self-peptide and tested for their proliferative responses and cytokine production. FIG. 6A shows that LNC from mice injected with DNA coding for the PLP139-151 self-peptide had lower proliferative responses when compared with the LNC from control animals (p<0.01). FIG. 6(B) shows that, when stimulated with the PLP 139-151, LNC from mice treated with the self-vector containing DNA coding for the PLP139-151 self-peptide secrete lower levels of IL-2 and □-interferon in comparison with control groups. A ribonuclease protection assay on mRNA isolated from brain tissue was used to evaluate the levels of cytokine mRNA transcripts in inflamed brain. FIG. 6(C) reveals a reduction in mRNA levels of □-interferon and IL-15 in mice treated with the self-vector comprising DNA encoding the PLP139-151 self-peptide. Therefore, a correlation between low incidence of clinical disease, reduced cellular responses, and low levels of IL-2, IL-15 and □-interferon is evident in the PLP 139-151 DNA treated mice. The relative expression levels of cytokine mRNA's bands shown in FIG. 6(C) were measured by densitometry. In order to correct for loading differences, the values were normalized according to the level of expression of the housekeeping gene, GAPDH, within each sample. Densitometric analysis confirmed reduction of expression level of the tested cytokines in brains of mice treated with the self-vector containing DNA encoding the PLP 139-151 self-peptide compared to pTargeT and a self-vector containing DNA encoding PLP139-151 (L/R) self-peptide.

### EXAMPLE 3

### Treatment of an Animal Model of Multiple Sclerosis Using IMS in Combination with DNA Encoding Multiple Self-Proteins

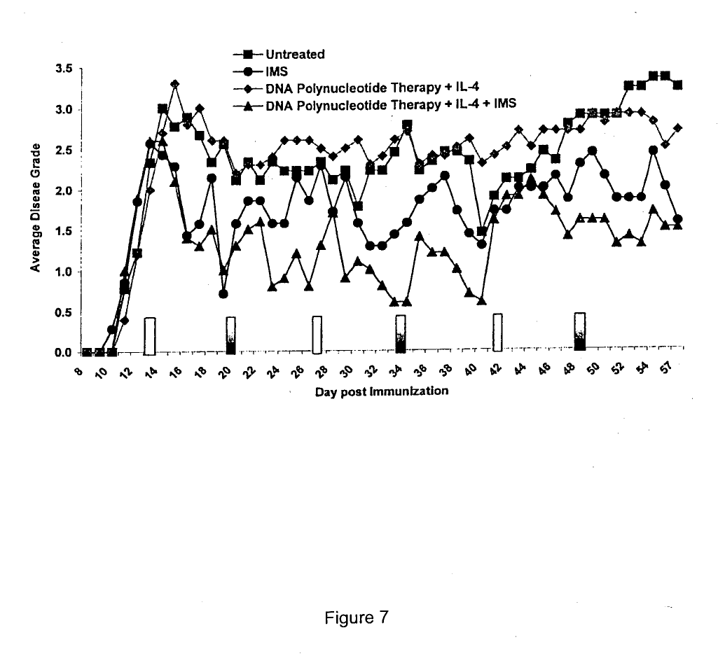

A DNA polynucleotide therapy composed of full-length cDNAs encoding the four major components of myelin, MBP, MAG, MOG, and PLP treated relapsing disease in the EAE animal model when given after initial disease onset. Moreover, with the addition of DNA encoding IL-4 to the myelin DNA polynucleotide therapy, the efficacy of treatment is further enhanced by a decrease in relapse rate. However, despite the reduction in relapses, the overall disease severity is still comparable to the control group. In a separate series of studies, SJL/J mice were immunized subcutaneously for disease induction with PLP139-151 peptide in complete Freund's adjuvant (CFA) and concurrently were administered an IMS resuspended in phosphate buffered saline intraperitoneally as a single injection. Mice treated with just a single injection of inhibitory IMS exhibited an overall decreased disease severity as compared to PBS-treated and stimulatory CpG-ODN treated mice (FIG. 7).

### EXAMPLE 4

### Ordered Peptides for Immunomodulation Based on MHC TCR Binding Motifs

The region between the amino acids 85 to 99 of myelin basic protein (MBP) contain the imrnunodominant epitope for T cells and autoantibodies in MS brain lesions. The main region of MBP recognized by T cells and autoantibodies, found in MS brain, is the core motif, {SEQ ID NO:8} HFFK, from MBPp87-99 in patients who are HLA-DRB1*1501 DQB1*0602 (HLA DR2).

Previously, we have compared the structural requirements for autoantibody recognition to those of T cell clones reactive to MBP p87-99. Anti-MBP antibodies were affinity-purified from CNS lesions of 12 post-mortem cases studied. The MBP p87-99 peptide was immunodominant in all cases and it inhibited autoantibody binding to MBP by more than 95%. Residues contributing to autoantibody binding were located in a 10-amino acid segment p86-95 ({SEQ ID NO:9} VVHFFKNIVT) that also contained the MHC T-cell receptor contact residues for T cells recognizing MBP in the context of DRB1*1501 and DQB1*0602. In the epitope center, the same residues, {SEQ ID NO: 10} VHFFK, were important for T cell binding and MHC recognition. Recently, the crystal structure of HLA-DR2 with MBPp85-99 was solved, confirming the prediction that K91 is the major TCR contact residue, while F90 is a major anchor into the hydrophobic P4 pocket of the MHC molecule.

Peptides were synthesized that contained repetitive sequences of three amino acids ordered to bind the pockets existing in MS related MHC molecules and therefore to interfere with the activation of pathogenic T cells. One of those predicted sequences ({SEQ ID NO:4} EYYKEYYKEYYK), was effective in preventing and treating experimental autoimmune encephalomyelitis in Lewis rats, an animal model of Multiple Sclerosis.

Materials and Methods

Animals. Female Lewis rats (6-8 weeks old), were purchased from Harlan Sprague Dawley (Indianapolis, Ind.)

Peptides. For immunization and disease reversal, peptides were synthesized on a peptide synthesizer (model 9050: MilliGen, Burlington, Mass.) by standard 9-fuorenylmethoxycarbonyl chemistry. Peptides were purified by HPLC. Structure was confirmed by amino acid analysis and mass spectroscopy. Peptides used for the experiments were: {SEQ ID NO:11} ENPVVHFFKNIVTPR (MBPp85 99), {SEQ ID NO:4} EYYKEYYKEYYK, {SEQ ID NO:5} KYYKYYKYYKYY.

EAE induction. Synthetic peptide MBPp85-99 was dissolved in PBS to a concentration of 2 mg/ml and emulsified with and equal volume of Incomplete Freund's Adjuvant (IFA), supplemented with 4 mg/ml heat-killed Mycobacterium tuberculosis H37Ra (Difco Laboratories, Detroit, Mich.). Rats were injected subcutaneously with 0.1 mi of the peptide emulsion. Experimental animals were scored as follows: 0, no clinical disease; 1, tail weakness or paralysis; 2, hind limb weakness; 3, hind limb paralysis; 4, forelimb weakness or paralysis; 5, moribund or dead animal.

EAE treatment. Rats previously immunized with MBPp85-99 for EAE induction were scored from day eight after peptide injection. On the day of mean disease onset, animals were injected intraperitoneally with a solution of 0.5 mg of peptide in PBS (one dose of 0.25 ml).

Results

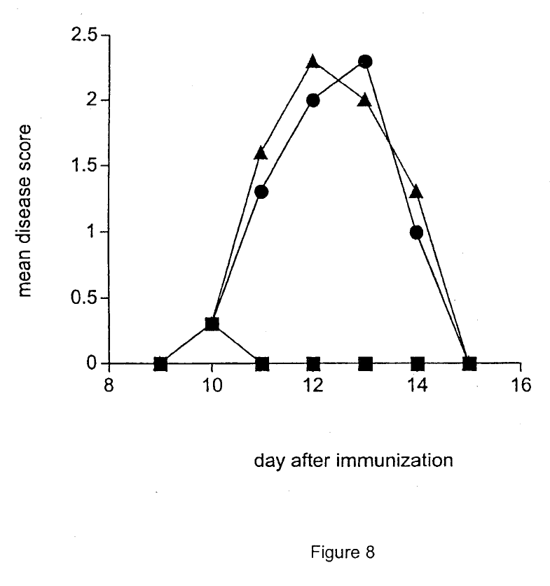

Injection of ordered peptides containing TCR-MHC binding motifs reverse the development of EAE. In order to test the potential of the predicted sequences to revert the development of ongoing EAE we delivered a single dose of a PBS solution containing 0.5 mg of peptide in 0.25 ml. As seen in FIG. 8, this dose is enough to treat the ongoing disease, when compared with the control groups.

### EXAMPLE 5

### Combination of Atorvostatin and Polynucleotide Therapy for Treatment of an Animal Model of Multiple Sclerosis

PLP self-vector. A polynucleotide encoding the PLP self-protein was constructed using standard techniques. Briefly, the mouse full-length PLP gene was cloned from a mouse cDNA library and cloned into the multiple cloning site of a mammalian expression vector, pVAX1 (Invtirogen, Carlsbad, Calif.). Large scale production and purification of the plasmid DNA was done by standard techniques using a commercial plasmid purification service (Elim Biopharmaceuticals, Hayward, Calif.).

Polynucleotide therapy protocol. Experimental animals were injected in each quadraceps with 0.05 ml of 0.25% bupivacaine-HCl (Sigma, St. Louis, Mo.) in PBS. Two to three days later, mice were injected with 0.05 mg of plasmid DNA (0.25 mg/ml in PBS), intramuscluarly.

Atorvastatin administration. Atorvastatin (Pfizer Inc., Groton, Conn.) (prescription formulation) was brought into suspension in PBS. Atorvastatin was administered orally in 0.5 ml (0.04 mg/ml for 1 mg/kg dose or 0.4 mg/ml for 10 mg/kg dose) once daily using 20 mm feeding needles (Popper and Sons Inc, New Hyde Park, N.Y.). PBS was administered as control.

EAE induction. PLP139-151 peptide was dissolved in PBS to a concentration of 2 mg/ml and emulsified with an equal volume of Incomplete Freund's Adjuvant supplemented with 4 mg/ml heat-killed mycobacterium tuberculosis H37Ra (Difco Laboratories, Detroit, Mich.). Mice were injected subcutaneously with 0.1 ml of the peptide emulsion. Experimental animals were scored as follows: 0=no clinical disease; 1=tail weakness or paralysis; 2=hind limb weakness; 3=hind limb paralysis; 4=forelimb weakness or paralysis; 5=moribund or dead animal. Mice induced for EAE with this method develop disease in such a manner that some of the mice develop relapsing-remitting EAE, whereas other mice develop a chronic progressive disease.

To determine whether injection of DNA encoding PLP sequences in combination with atorvastatin is effective in treating on-going EAE, mice were induced for EAE disease as above and allowed to reach peak acute disease. On approximately day 17 after the induction of EAE, mice were randomly distributed into various treatment groups. Mice which received atorvastatin only were treated daily with oral administration of atorvastatin at either 1 mg/kg or 10 mg/kg. Mice which received a combination of atorvastatin or DNA were administered atorvastatin orally on a daily basis at either 1 mg/kg or 10 mg/kg, along with DNA encoding PLP intramuscularly on a once weekly basis at a dose of 0.05 mg per mouse. Control mice were administered PBS orally on a daily basis. EAE clinical disease was then followed for a total of approximately 45 days from the induction of EAE.

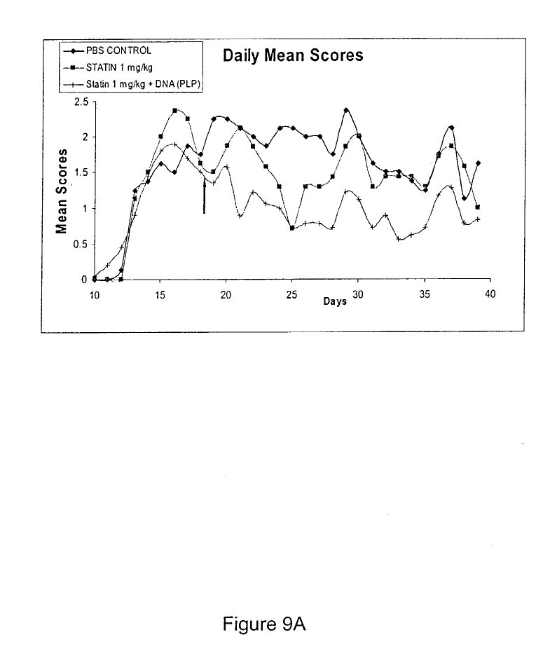

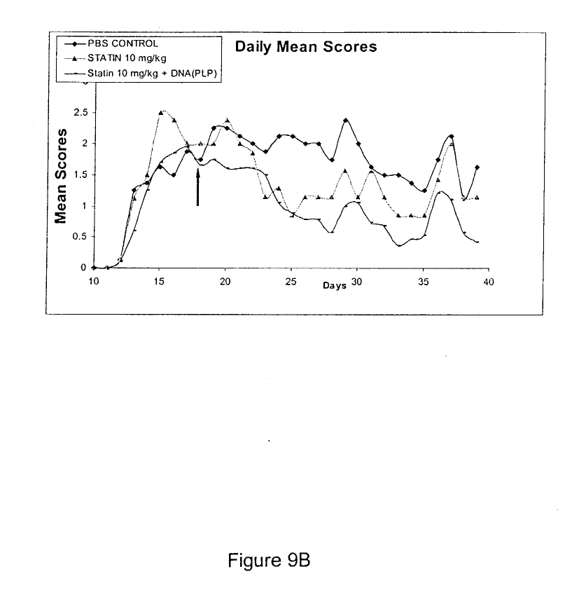

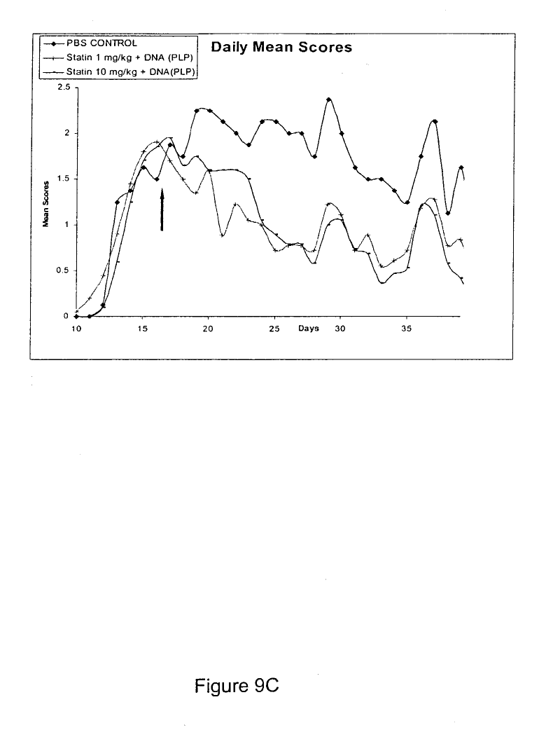

The mean peak disease severity was reduced significantly in those mice that received both atorvastatin and DNA for PLP (FIG. 9) as compared to both the control PBS administered mice as well as the mice administered atorvastatin only. Even more significantly, the DNA provided as equal a benefit at both the 1 and 10 mg/kg dose of atorvastatin. This suggests that the DNA can provide a benefit even at a lower, more tolerated dose of atovastatin. Further these results suggest that this combination therapy can be used to treat both relapsing-remitting as well as chronic progressive disease.

### EXAMPLE 6

### Combination of Statin and Polynucleotide Therapy for the Treatment of Relapsing-Remitting and Chronic-Progressive Human Multiple Sclerosis

Polynucleotide therapy to treat human multiple sclerosis is carried out as follows. A self-vector is constructed comprising the cytomegalovirus or another effective transcriptional promoter; a polyadenylation signal derived from the SV40 large T antigen, bovine growth hormone, or another effective polyadenylation signal sequence known to the ordinarily skilled artisan; and, a kanamycin or other FDA-acceptable resistance gene to enable efficient growth of the plasmid.

DNA sequences encoding one or more of the human myelin self-proteins are cloned into the DNA self-vector. DNA encoding those myelin self-proteins targeted by the autoimmune response in MS patients including myelin basic protein (MBP), proteolipid protein (PLP), myelin-associated oligodendrocytic basic protein (MOBP) is cloned into the self-vector. Selection of a particular autoantigen for inclusion in polynucleotide therapy is based on various factors using the teaching of this invention and includes such factors as the presence of pathogenic autoantibodies in a subject. In one embodiment each myelin self-protein is encoded in a separate or distinct self-plasmid. In another embodiment, DNA encoding several myelin self-proteins are encoded sequentially in a single self-plasmid utilizing internal ribosomal re-entry sequences (IRESs) or other methods to express multiple proteins from a single plasmid DNA. The DNA expression self-plasmids encoding the myelin proteins are prepared and isolated using commonly available techniques for isolation of plasmid DNA such as those commercially available from Qiagen Corporation. The DNA is purified free of bacterial endotoxin for delivery to humans as a therapeutic agent. In one embodiment self-vector DNA encoding only MBP is administered to treat patients with multiple sclerosis. In another embodiment multiple self-plasmids encoding two or more myelin self-protein(s), -polypeptide(s) or -peptide(s) is administered. Therapeutically effective amounts of the self-vector comprising a polynucleotide encoding one or more self-polypeptide(s) is administered in accord with the teaching of this invention. For example, therapeutically effective amounts of self-vector are in the range of about 0.001 micrograms to about 1 gram. A preferred therapeutic amount of self-vector is in the range of about 10 micrograms to about 5 milligrams. A most preferred therapeutic amount of self-vector is in the range of about 0.025 mg to 5 mg. The polynucleotide therapy is delivered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the self-polypeptide(s) being administered and such other factors as would be considered by the ordinary treating physician.

In one embodiment the DNA is delivered by intramuscular injection. In another embodiment the DNA is delivered as an inhaled agent, intranasally, orally, subcutaneously, intradermally, intravenously, impressed through the skin, or attached to particles or beads delivered to or through the dermis. Such particles or beads can be gold, other metals, polystyrene, or other particles. In one embodiment, the DNA is formulated in phosphate buffered saline with physiologic levels of calcium (0.9 mM). Alternatively the DNA is formulated in solutions containing higher quantities of Ca++, between 1 mM and 2M. In another embodiment, the DNA is formulated with other cations such as zinc, aluminum, and others. The DNA could also be formulated with a cationic polymer, with a cationic liposome, or in other liposomes. The DNA could also be delivered encoded in a viral vector, viral particle, or bacterium.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

Human MS patients treated with the disclosed combination of statin and polynucleotide therapy will be monitored for disease activity based on the overall disability score, the number of clinical relapses, and MRI monitoring for the number of new gadolinium-enhancing lesions and the volume of the enhancing lesions. This combination therapy will be used to treat both relapsing-remitting as well as chronic-progressive MS.

### EXAMPLE 7

### Combination of Statin and Polynucleotide Therapy Comprising Administration Of DNA Encoding the Self-Peptide of the Insulin 13 Chain for Prevention of Insulin Dependent Diabetes Mellitus

NOD mice develop spontaneous autoimmune diabetes, and share many clinical, immunological, and histopathological features with human IDDM. Polynucleotide therapy is performed with a self-vector comprising a DNA encoding the self-peptide of amino acids 9-23 of the insulin B chain, the immunodominant epitope of insulin is administered to NOD mice. The control is a vector comprising DNA encoding a corresponding peptide on the A chain of insulin. Overlapping oligonucleotide primers encoding the self-peptide are inserted into an expression self-cassette, PcDNA. Treatment with self-vector encoding the self-peptide insulin B (9-23) (insB-PcDNA) is predicted to effectively protect animals from developing diabetes. The nucleotide sequence of the insulin A (+) strand is 5′-CCGGAATTCGCCATGTGCACGTCAATCTGTTCACTGTACCAGCTAGAGAACTACTGCAACTAGTCTAQGAGC-3′ (SEQ ID NO: 74); the sequence of the insulin B (+) strand is 5′-CCGGAATTCGCCATGAGCCACCTAGTAGAAGCACTATACCTCGTATGCGGCGAACGAGGTTAGTCTAGAGC-3′ (SEQ ID NO: 75). These polynucleotides are designed to incorporate EcoRI and XbaI restriction sites for cloning. The products are cloned into the multiple cloning region of an appropriate expression vector such as PcDNA3.1+ (Invitrogen, Carlsbad, Calif.). Purification of the self-plasmid DNA is carried out using Qiagen Endo-free Mega-prep kits (Qiagen, Valencia, Calif.).

Three- to four-week-old female NOD mice are purchased from Taconic Farms (Germantown, N.Y.). Experimental animals are injected at 3 to 4 weeks of age in the quadricep with 0.1 ml of 0.25% bupivicaine-HCL (Sigma, St. Louis, Mo.) in PBS (0.05 ml per quadricep). Two days following, mice are injected with 0.05 ml of plasmid DNA at 1.0 mg/ml in each quadricep. The plasmid DNA is injected two more times at ten-day intervals.

In combination with the above described polynucleotide therapy, 3-hydroxy-3-methylglutaryl coenzyme A (HMG-CoA) reductase inhibitors or “statins” are administered orally at 1 mg/kg or 10 mg/kg as described in Example 5.

Mice are tested weekly for glucosuria by Chemstrip (Boehringer Mannheim Co., Indianapolis, Ind.), and diabetes is confirmed by plasma glucose measurement using the One Touch II meter (Johnson & Johnson, Milpitas, Ca). Animals having repeated plasma glucose levels greater than 250 mg/dl are considered diabetic. The pancreata are removed from experimental and control animals, fixed in 10% formaldehyde, and embedded in paraffin. Thin sections at three levels, 50 □m apart, are cut for staining with hematoxylin and eosin. The severity of infiltration is assessed by light microscopy. In addition the antigen-specific response in the pancreatic lymph nodes are examined by ELISA and RT-PCR for such cytokines as IL-4, IL-10, IFN-γ, and TGF-β.

### EXAMPLE 8

### Combination of Statin and Polynucleotide Therapy Comprising Administration of DNA Encoding the Self-Polypeptide Insulin and Self-Proteins Glutamic Acid Decarboxylase and Tyrosine Phosphatase for Treatment of Insulin Dependent Diabetes Mellitus

NOD mice are treated with polynucleotide therapy comprising DNA encoding the whole pro-insulin polypepide along with DNA encoding glutamic acid decarboxylase (GAD) 65 kDa or the islet tyrosine phosphatase IA-2. The cDNAs encoding proinsulin, GAD 65, and IA-2 is isolated and cloned into the expression self-cassette pTARGET vector. The DNA is purified using Qiagen Endo-free Mega-prep kits (Qiagen, Valencia, Calif.).

NOD mice are injected at 3 to 4 weeks of age in the quadricep with 0.1 ml of 0.25% bupivicaine-HCL (Sigma, St. Louis, Mo.) in PBS (0.05 ml per quadricep). Two days following, mice are injected with 0.05 ml of each self-plasmid DNA at 1.0 mg/ml in phosphate buffered saline with 0.9 mM calcium in each quadricep. The plasmid DNA is injected two more times at ten-day intervals.

In combination with the above described polynucleotide therapy, 3-hydroxy-3-methylglutaryl coenzyme A (HMG-CoA) reductase inhibitors or “statins” are administered orally at 1 mg/kg or 10 mg/kg as described in Example 5.

Mice are tested weekly for glucosuria by Chemstrip (Boehringer Mannheim Co., Indianapolis, Ind.), and diabetes is confirmed by plasma glucose measurement using the One Touch II meter (Johnson & Johnson, Milpitas, Ca). Animals having repeated plasma glucose levels greater than 250 mg/dl are considered diabetic.

### EXAMPLE 9

### Combination of Statin and Polynucleotide Therapy Comprising Administration of DNA Encoding the Self-Polypeptide Insulin and/or Self-Proteins Glutamic Acid Decarboxylase and Tyrosine Phosphatase for Treating and Reversing Overt Hyperglycemia in Established Insulin Dependent Diabetes Mellitus

NOD mice are identified to have overt clinical diabetes based on glucosuria detected using Chemstrip (Boehringer Mannheim Co., Indianapolis, Ind.) analysis of urine, with confirmation of diabetes by plasma glucose measurement using the One Touch II meter (Johnson & Johnson, Milpitas, Ca). NOD mice with overt clinical diabetes are treated with polynucleotide therapy comprising DNA encoding the self-peptide insulin B (9-23) (insB-PcDNA) or DNA encoding the whole pro-insulin polypepide along with DNA encoding glutamic acid decarboxylase (GAD) 65 kDa or the islet tyrosine phosphatase IA-2 as in Examples 7 and 8.

In combination with the above described polynucleotide therapy, 3-hydroxy-3-methylglutaryl coenzyme A (HMG-CoA) reductase inhibitors or “statins” are administered orally at 1 mg/kg or 10 mg/kg as described in Example 5.

Mice are tested weekly for glucosuria by Chemstrip (Boehringer Mannheim Co., Indianapolis, Ind.), and diabetes is confirmed by plasma glucose measurement using the One Touch II meter (Johnson & Johnson, Milpitas, Ca). Animals having repeated plasma glucose levels greater than 300 mg/dl are considered to have failed treatment.

### EXAMPLE 10

### Combination of Statin and Polynucleotide Therapy for the Treatment of Human Insulin Dependent Diabetes Mellitus

A self-plasmid is constructed comprising of DNA encoding a human islet cell self-proteins such as the tyrosine phosphatase IA-2, glutamic acid decarboxylase (GAD) (either the 65 kDa and 67 kDa forms), preproinsulin, or islet cell antigen 69 KDa (ICA69). The DNA is isolated using PCR and cloned into the expression self-cassette as described previously. Therapeutically effective amounts of the self-vector comprising a polynucleotide encoding one or more self-polypeptide(s) is administered in accord with the teaching of this invention. For example, therapeutically effective amounts of self-vector are in the range of about 0.001 micrograms to about 1 gram. A preferred therapeutic amount of self-vector is in the range of about 10 micrograms to about 5 milligrams. A most preferred therapeutic amount of self-vector is in the range of about 0.025 mg to about 5 mg. The DNA therapy is delivered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the self-polypeptide(s) being administered and such other factors as would be considered by the ordinary treating physician. In the preferred embodiment the DNA is delivered by intramuscular injection. Alternatively, the DNA self-vector is delivered as an inhaled agent, intranasally, orally, subcutaneously, intradermally, intravenously, impressed through the skin, and in the case of treatment of IDDM attached to gold particles delivered by gene gun to or through the dermis. The DNA is formulated in phosphate buffered saline with physiologic levels of calcium (0.9 mM). Alternatively the DNA is formulated in solutions containing higher quantities of Ca++, between 1 mM and 2M. The DNA is formulated with other cations such as zinc, aluminum, and others.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

Human diabetes patients treated with the disclosed combination of statin and polynucleotide therapy will be monitored for disease activity based on decreased requirement for exogenous insulin, alterations in serum autoantibody profiles, decrease in glycosuria, and decrease in diabetes complications such as cataracts, vascular insufficiency, arthropathy, and neuropathy.

### EXAMPLE 11

### Combination of Statin and Polynucleotide Therapy Comprising Adminstration of DNA Encoding Self-Protein Type II Collagen for Prevention of Autoimmune Synovitis and Rheumatoid Arthritis

RA arises from pathogenic T cells that evade mechanisms promoting self-tolerance. Collagen-induced arthritis (CIA) in mice is a model of T cell-mediated autoimmunity that shares many features with RA, including synovitis and bony erosions that histologically resemble those in RA. The relapsing model of CIA has clinical relapses and remissions of inflammatory erosive synovitis in a similar fashion to that observed in human RA patients (Malfait et al, Proc Natl Acad Sci USA, 97:9561-6, 2000). CIA is induced by injecting genetically susceptible strains of mice with type II collagen (CII) in complete Freund's adjuvant.

The cDNA encoding murine type II collagen is isolated using the polymerase chain reaction. Additional synovial self-proteins such as collagens type IV and IX, and heat shock protein 65 may be included in the polynucleotide therapy. DNA encoding the described peptides is obtained using oligonucleotide primers to amplify the relevant fragments of DNA by PCR from murine CII cDNA. An in frame methionine start of translation site as well as Xho I and Xba I restriction endonuclease sites are incorporated within the oligonucleotide primers. The PCR-generated DNA fragments are cloned into the Xho I and Xba I restriction endonuclease sites of an expression self-cassette such as in the pTARGET Vector (Promega, Madison, Wis.), a mammalian expression vector driven by the CMV promoter. The isolated clones are sequenced to confirm that the desired DNA sequence has been produced.

Male DBA/1LacJ (H-2q) mice between 6-9 weeks of age at the start of the experiment are used. 100 μg of each of the purified self-plasmids comprising DNA encoding the synovial joint self proteins are injected intramuscularly into the tibialis anterior muscle 3 times at weekly intervals prior to induction of disease for the prevention of CIA experiments, or following onset of clinical CIA in the treatment of relapsing CIA experiments. Such DNA therapy is initiated following injection at the site of administration with bupivicane, cardiotoxin, or another pre-conditioning agent, or without such an agent.

In combination with the above described polynucleotide therapy, 3-hydroxy-3-methylglutaryl coenzyme A (HMG-CoA) reductase inhibitors or “statins” are administered orally at 1 mg/kg or 10 mg/kg as described in Example 5.

After the combination treatment, mice are challenged intradermally at the base of the tail with 100 μg purified heterologous CII protein in complete Freund's adjuvant (CFA) to induce acute CIA, or homologous CII in CFA to induce relapsing CIA (Malfait et al, Proc. Natl. Acad. Sci. USA, 97:9561-66). The mice are followed daily for 12 weeks for clinical evidence of CIA based on the visual scoring system (Coligan et al., John Wiley and Sons, Inc 15.5.1-15.5.24, 1994): 0, no evidence of erythema and swelling; 1, erythema and mild swelling confined to the mid-foot (tarsals) or ankle joint; 2, erythema and mild swelling extending from the ankle to the mid-foot; 3, erythema and moderate swelling extending from the ankle to the metatarsal joints; and 4 erythema and severe swelling encompassing the ankle, foot and digits. The clinical score for each animal is the sum of the visual score for each of its four paws. Histologic analysis is performed on joints from mice that develop clinical arthritis. The first paw from the limb with the highest visual score is decalcified, sectioned, and stained with hematoxylin and eosin as previously described (Williams et al., Proc Natl Acad Sci USA 91: 2762-2766, 1994). The stained sections are examined for lymphocytic infiltration, synovial hyperplasia and erosions as previously described (Williams et al., Proc Natl Acad Sci USA 91: 2762-2766, 1994).

### EXAMPLE 12

### Combination of Statin and Polynucleotide Therapy Comprising Adminstration of DNA Encoding Self-Protein Type II Collagen for Treatment of Established Autoimmune Synovitis and Rheumatoid Arthritis

Animals with established ongoing CIA are treated with self-vector DNA encoding CII, BiP, and/or GP-39 to reverse established ongoing CIA. The mice are followed daily for 12 weeks for clinical evidence of CIA based on the visual scoring system (Coligan et al., John Wiley and Sons, Inc 15.5.1-15.5.24, 1994): 0, no evidence of erythema and swelling; 1, erythema and mild swelling confined to the mid-foot (tarsals) or ankle joint; 2, erythema and mild swelling extending from the ankle to the mid-foot; 3, erythema and moderate swelling extending from the ankle to the metatarsal joints; and 4 erythema and severe swelling encompassing the ankle, foot and digits. The clinical score for each animal is the sum of the visual score for each of its four paws. Histologic analysis is performed on joints from mice that develop clinical arthritis. The first paw from the limb with the highest visual score is decalcified, sectioned, and stained with hematoxylin and eosin as previously described (Williams et al., Proc Natl Acad Sci USA 91: 2762-2766, 1994). The stained sections are examined for lymphocytic infiltration, synovial hyperplasia and erosions as previously described (Williams et al., Proc Natl Acad Sci USA 91: 2762-2766, 1994). Treatment with a combination of statin and self DNA encoding CII, BiP, GP-39 and/or additional proteins present in synovial joints will reduce the number of clinical relapses of synovitis and reduce the severity of arthritis based on the visual scoring system.

### EXAMPLE 13

### Combination of Statin and Polynucleotide Therapy for the Prevention of, or Treatment of, Human Rheumatoid Arthritis and Other Autoimmune Diseases Targeting Joints

A self-plasmid is constructed comprising of DNA encoding human self-proteins, such as proteins expressed in synovial joints including type II collagen, BiP, gp39, collagen type IV, glucose-6-phosphate isomerase and/or fibrin. The DNA is isolated using PCR and cloned into the expression self-cassette as described previously. 100 μg of plasmid DNA is administered in phosphate buffered saline with calcium intramuscularly on a monthly basis. It is also possible to administer the DNA in different dosing regimens, formulated in different buffers, or via different routes of administration as discussed in the above examples.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

Humans with new-onset or ongoing RA, diagnosed based on the American College of Rheumatology Criteria (4/7 criteria required for diagnosis: (i) symmetrical polyarthritis, (ii) involvement of the MCPs, PIPs, or wrists, (iii) involvement of more than 3 different joint areas, (iv) joint erosions on X rays of hands or feet, (v) positive rheumatoid factor test, (iv) greater than 1 hour of morning stiffness, and (vii) nodules on extensor surfaces) are treated with self polynucleotides encoding type II collagen, BiP, gp39, collagen type IV, glucose-6-phosphate isomerase and/or fibrin in combination with a statin. The efficacy of the combination therapy for RA is monitored based on the fraction of patients with a reduction in their tender and swollen joint count by greater than 20% (an American College of Rheumatology 20% Response, ACR20), 50% (ACR50), and 70% (ACR70). Additional measures for human RA include inflammatory markers (including ESR and CRP) reduction in steroid usage, reduction in radiographic progression (including erosions and joint space narrowing) and improvement in disability status scores (such as the Health Assessment Questionnaire—HAQ). Changes in autoantibody titers and profiles will also be monitored. An identical approach will be used for related arthritides such as psoriatic arthritis, reactive arthritis, Reiter's syndrome, Ankylosing spondylitis, and polymyalgia rheumatica.

Recent studies have suggested that certain autoantibodies with high specificity for rheumatoid arthritis (e.g., BiP, anti-citrulline antibodies, anti-filaggrin antibodies) may precede the clinical diagnosis by months, or even years. This raises the possibility that patients could be identified prior to disease onset, and effectively treated using a preventative polynucleotide therapeutic. Healthy, asymptomatic patients will be screened for the presence of a diagnostic autoantibody, including but not limited to one of the serological tests described above. Patients with a positive test will be treated with a combination of a statin and a polynucleotide therapeutic as described above and in other examples, in an attempt to prevent disease onset and severity. Subsequent diagnosis and response will be monitored using the above criteria.

### EXAMPLE 14

### Combination of Statin and Polynucleotide Therapy for Treating Human Autoimmune Uveitis

Using PCR human S-antigen, interphotoreceptor retinoid binding protein (IRBP), rhodopsin, and recoverin are isolated and cloned into a DNA expression self-cassette as described in Example 5. A self-plasmid is constructed comprising of DNA encoding a polynucleotide encoding one or more of the self-polypeptide(s) selected from the group consisting of human S-antigen, interphotoreceptor retinoid binding protein, rhodopsin and recoverin. Therapeutically effective amounts of the self-vector comprising polynucleotide encoding one or more self-polypeptide(s) is administered in accord with the teaching of this invention. For example, therapeutically effective amounts of self-vector are in the range of about 0.001 micrograms to about 1 gram. A preferred therapeutic amount of self-vector is in the range of about 10 micrograms to about 5 milligrams. A most preferred therapeutic amount of self-vector is in the range of about 0.025 mg to about 5 mg. The DNA therapy is delivered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the self-polypeptide(s) being administered and such other factors as would be considered by the ordinary treating physician.

In a preferred embodiment the DNA is delivered by intramuscular injection. Alternatively, the DNA self-vector is delivered as an inhaled agent, intranasally, orally, subcutaneously, intradermally, intravenously, impressed through the skin, and in the case of treatment of autoimmune uveitis attached to gold particles delivered to or through the dermis. In another embodiment, the DNA is formulated in phosphate buffered saline with physiologic levels of calcium (0.9 mM). Alternatively the DNA can be formulated is solutions containing higher quantities of Ca++, between 1 mM and 2M. The DNA could be formulated with other cations such as zinc, aluminum, and others.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

### EXAMPLE 15

### Combination of Statin and Polynucleotide Therapy for Prevention of Primary Biliary Cirrhosis, and Treatment of Established Primary Biliary Cirrhosis

DNA encoding human PDC-E2 and -E3 is isolated using PCR and cloned into the expression self-cassette of a suitable mammalian expression vector, amplified in E. coli, and purified using an endotoxin-free plasmid purification method. Polynucleotide therapy comprising DNA encoding self-protein(s) PDC-E2 and -E3 is administered to humans with established PBC. A vector comprising DNA encoding a cytokine, such as IL-4, may be administered with the self-vector.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

Patients with PBC, or at risk to develop PBC, can be efficiently diagnosed by identifying serum autoantibodies directed against mitochondrial proteins such as the pyruvate dehydrogenase complex. Asymptomatic human patients will be tested using available serlogic tests such as ELISA, Western blot, or protein array for the presence of diagnostic autoantibodies. Patients with a positive serological test will be treated prophylactically with polynucleotide therapy as described above to prevent disease onset. The efficacy of the combination therapy for PBCs in humans is determined by measuring serial liver function tests including bilirubin, alkaline phosphatase, alanine amino transferase (ALT), and aspartate aminotransferase (AST), as well as the delay in time to progression to liver failure. Following percutaneous liver biopsy, liver histology is evaluated by haematoxylin & eosin stain and periodic acid Schiff. Bile duct abnormalities, necro-inflammatory changes in portal tracts and granulomatous infiltration are also examined for evidence of disease activity. Serum autoantibody profiles will also be analyzed.

### EXAMPLE 16

### A Method to Treat Multiple Sclerosis and Other Autoimmune Diseases with a Combination of Statin and DNA Encoding Osteopontin

Osteopontin is a pleiotrophic molecule recently identified to play pathogenic roles in multiple sclerosis and its animal model, EAE. Osteopontin may also play central roles in inflammatory arthritis and other human autoimmune diseases. Treatment of mice with DNA encoding the self protein osteopontin induces an anti-osteopontin immunoglobulin response in the host that inhibits the detrimental impact of osteopontin in perpetuating the disease.

In humans with multiple sclerosis osteopontin-self-vector therapy is initiated following diagnosis. In combination with the this polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose. Efficacy is monitored based on induction of anti-osteopontin antibodies in the patient with multiple sclerosis, as measured by ELISA analysis. Efficacy is further demonstrated based on reduction in the number and size of lesions on brain MRI scanning, reduction of the number of disease relapses (episodes of clinical paralysis), and slowing of progression to disability.

### EXAMPLE 17

### Combination of Statin and Polynucleotide Therapy Comprising Adminstration of DNA Encoding a Library of Self-Proteins Expressed in a Organ or Tissue Targeted by the Autoimmune Response

Another strategy for the treatment of autoimmunity is to administer DNA encoding many or all of the self-proteins present within a tissue or organ under immune attack. cDNA expression libraries contain cDNA encoding many or the vast majority of the self-proteins expressed in a specific tissue, organ, or cell type. Such cDNA expression libraries are generated in the self-vector to enable expression of the polypeptides they encode upon administration to a host. Animals and humans with established multiple sclerosis are treated with self-vector encoding a library of cDNA expressed in oligodendrocytes in the brain. Animal and humans with rheumatoid arthritis are treated with self-vector encoding a library of cDNA expressed in synovial joints which are the target of the autoimmune response in rheumatoid arthritis. Animals and humans with autoimmune diabetes are treated with self-vector encoding a library of cDNA expressed in beta cells of the pancreas. Self-vector encoding cDNA expressed in the beta cells of the pancreas can also be utilized to prevent development of clinical diabetes in individuals identified to have a high risk of developing autoimmune diabetes. Alternatively, instead of using the whole cDNA library a large subset of the cDNA expression library encoded in self-vector can be used to treat autoimmunity.

In combination with the above described polynucleotide therapy, 3-hydroxy-3-methylglutaryl coenzyme A (HMG-CoA) reductase inhibitors or “statins” are administered orally at the currently approved doses for hypercholesterolemia. For example, patients are administered atorvastatin at a dose range of 10 to 80 mg once daily, with a preferred dose of 40 mg once daily as a maintenance dose.

### EXAMPLE 18

### Combination of Statin and Ordered Peptide for Treatment of Multiple Sclerosis

It has previously been shown that certain ordered peptides that contained repetitive sequences of three amino acids ordered to bing the pockets existing in MS related MHC molecules could reduce disease severity in animal models of MS. For example, it was demonstrated that a particular sequence of amino acids (EYYKEYYKEYYK) based on the MBP p87-99 sequence found to be immunodominant in MS patients, could effectively prevent and treat EAE in Lewis rats.

In the combination approach of this invention, ordered peptides such as the EYYKEYYKEYYK are administered subcutaneously to the MS patient either daily, semi-weekly, weekly, semi-monthly, monthly, or occasionally in combination with a statin administered as described in Example 6.

Human MS patients treated with the disclosed combination of statin and ordered peptide will be monitored for disease activity based on the number of clinical relapses and MRI monitoring for the number of new gadolinium-enhancing lesions and the volume of the enhancing lesions.

### EXAMPLE 19

### Combination of Statin and Immune Modulatory Sequences (IMS) for Treatment of Multiple Sclerosis

It has previously been shown that certain single-strand oligonucleotides with a phosphorothioate backbone called IMS's that preferentially contained the hexamer motif 5′-Purine-Purine-G-G-Pyrimidine-Pyrimidine-3′ or 5′-Purine-Pyrimidine-G-G-Pyrimidine-Pyrimidine-3′ could reduce disease severity in animal models of MS.

In the combination approach of this invention, IMS oligos are administered subcutaneously to the MS patient either daily, semi-weekly, weekly, semi-monthly, monthly, or occasionally in combination with a statin administered as described in Example 6.

Human MS patients treated with the disclosed combination of statin and IMS will be monitored for disease activity based on the number of clinical relapses and MRI monitoring for the number of new gadolinium-enhancing lesions and the volume of the enhancing lesions.

What is claimed is:

1. A method of treating an autoimmune disease, the method comprising:
co-administering to a patient suffering from the autoimmune disease an effective amount of a statin and an effective amount of an antigen-specific immunomodulatory agent.

2. The method of claim 1, wherein the antigen-specific immunomodulatory agent is a self-vector comprising a polynucleotide encoding a self-polypeptide associated with the autoimmune disease.

3. The method of claim 2, wherein the self-polypeptide is a self-protein or self-peptide.

4. The method of claim 1, wherein the antigen-specific immunomodulatory agent is a polypeptide.

5. The method of claim 4, wherein the polypeptide is a protein or peptide.

6. The method of claim 4, wherein the polypeptide is a derivative polypeptide.

7. The method of claim 4, wherein the polypeptide comprises a self-polypeptide associated with the disease.

8. The method of claim 4, wherein the polypeptide comprises amino acids corresponding to an autoantigenic epitope of a self-polypeptide associated with the disease.

9. The method of claim 8, wherein the amino acids corresponding to the autoantigenic epitope are randomized to form a random copolymer.

10. The method of claim 9, wherein the random copolymer is a peptide.

11. The method of claim 8, wherein the amino acids corresponding to the autoantigenic epitope are ordered, the polypeptide thereby comprising an ordered amino acid motif.

12. The method of claim 11, wherein the autoimmune disease is a demyelinating autoimmune disease and the ordered amino acid motif is [¹E²Y³Y⁴K]ₙ, where n is from 2 to 6.

13. The method of claim 1, wherein the autoimmune disease is selected from the group consisting of multiple sclerosis, insulin dependent diabetes mellitus (IDDM), rheumatoid arthritis, and autoimmune uveitis.

14. The method of claim 13, wherein the autoimmune disease is multiple sclerosis.

15. The method of claim 14, wherein the statin is selected from the group consisting of rosuvastatin, mevastatin, lovastatin, pravastatin, simvastatin, fluvastatin, atorvastatin, and cerivastatin.

16. The method of claim 15, wherein the statin is atorvastatin.

17. The method of claim 2, wherein the polypeptide encoded by the polynucleotide is selected from the group consisting of myelin basic protein (MBP), proteolipid protein (PLP), myelin associated glycoprotein (MAG), cyclic nucleotide phosphodiesterase (CNPase), myelin-associated oligodendrocytic basic protein (MBOP), myelin oligodendrocyte protein (MOG), and alpha-B crystalline.

18. The method of claim 2, wherein the autoimmune disease is insulin dependent diabetes mellitus (IDDM).

19. The method of claim 18, wherein the self-polypeptide encoded by the polynucleotide is selected from the group consisting of insulin, insulin B chain, preproinsulin, proinsulin, 65 kDA form of glutamic acid decarboxylase, 67 kDa form of glutamic acid decarboxylase, tyrosine phosphatase IA2 or IA-2b, carboxypeptidase H, a heat shock protein, glima38, 69 kDa form of islet cell antigen, p52, and islet cell glucose transporter (GLUT 2).

20. The method of claim 19, wherein the self-vector comprises a polynucleotide encoding one self-polypeptide.

21. The method of claim 20, wherein the self-polypeptide is preproinsulin.

22. The method of claim 20, wherein the self-polypeptide is insulin B chain 9-23.

23. The method of claim 2, wherein the autoimmune disease is rheumatoid arthritis.

24. The method of claim 23, wherein the polypeptide encoded by the polynucleotide is selected from the group consisting of type II collagen; hnRNP A2/RA33; Sa; filaggrin; keratin; cartilage proteins including gp39; collagens type I, III, IV, V, IX, XI; HSP-65/60; RNA polymerase; hnRNP-B1; hnRNP-D; and aldolase A.

25. The method of claim 2, wherein the autoimmune disease is autoimmune uveitis.

26. The method of claim 25, wherein the polypeptide encoded by the polynucleotide is selected from the group consisting of S-antigen, interphotoreceptor retinoid binding protein (IRBP), rhodopsin, and recoverin.

27. A method for treating an autoimmune disease, the method comprising:
co-administering to a patient suffering from the autoimmune disease an effective amount of a statin and an effective amount of an non-antigen-specific immunomodulatory agent.

28. The method of claim 27, wherein the non-antigen specific immunomodulatory agent is an immune modulatory sequence.

29. The method of claim 28, wherein the immune modulatory sequence is selected from the group consisting of
(a) 5′-Purine-Pyrimidine-[X]-[Y]-Pyrimidine-Pyrimidine-3′ and
(b) 5′-Purine-Purine-[X]-[Y]-Pyrimidine-Pyrimidine-3′,
wherein X and Y are any naturally occurring or synthetic nucleotide, except that X and Y cannot be cytosine-guanine.

30. The method of claim 27, wherein the non-antigen-specific immunomodulatory agent is osteopontin.

31. The method of claim 27, wherein the non-antigen-specific immunomodulatory agent is a self-vector comprising a polynucleotide encoding ostepontin.

32. The method of claim 27, wherein the autoimmune disease is selected from the group consisting of multiple sclerosis, insulin dependent diabetes mellitus (IDDM), rheumatoid arthritis, and autoimmune uveitis.

33. The method of claim 32, wherein the autoimmune disease is multiple sclerosis.

34. The method of claim 27, wherein the statin is selected from the group consisting of rosuvastatin, mevastatin, lovastatin, pravastatin, simvastatin, fluvastatin, atorvastatin, and cerivastatin.

35. The method of claim 34, wherein the statin is atorvastatin.
